(19) United States
(12) Patent Application Publication
(10) Pub. No.: US 2023/0023921 A1
(43) Pub. Date: Jan. 26, 2023

(54) MATERIAL SCIENCES APPLICATIONS FOR AN ORAL APPLIANCE FOR DELIVERY OF A MEDICAMENT

(71) Applicant: Emanate Biomedical, Inc., New York, NY (US)

(72) Inventors: Peter John Zegarelli, Sleepy Hollow, NY (US); Jarret Scott Fass, New York, NY (US); Nathaniel Schwartz, Brooklyn, NY (US)

(73) Assignee: Emanate Biomedical, Inc., New York, NY (US)

(21) Appl. No.: 17/783,994

(22) PCT Filed: Dec. 9, 2020
(86) PCT No.: PCT/US20/64027

§ 371 Date: Jun. 9, 2022

Related U.S. Application Data
(60) Provisional application No. 62/946,650, filed on Dec. 11, 2019.

(51) Int. Cl. A61C 19/06

(52) U.S. Cl. CPC A61C 19/063

(57) ABSTRACT

An oral appliance for delivering a medicament to an oral cavity is provided, the oral appliance having an interior surface and an exterior surface, the interior surface and/or exterior surface configured to contour a treatment area and deliver medicament thereto, the treatment area comprising soft tissue, hard tissue or a combination of soft tissue and hard tissue, at least a portion of the interior surface and/or exterior surface of the oral appliance being less, equal or greater in hardness, elasticity, flexibility, density, porosity, hydrophobicity, hydrophilicity relative to the treatment area. In some embodiments, the oral appliance is designed based on various material science properties to enhance medicament delivery and patient compliance.

### BACKGROUND

The present disclosure is generally related to the field of oral appliances for delivering a medicament to the oral cavity.

Medicaments may be delivered to patients by a variety of ways including oral, intravenous, intramuscular, inhalation, topical, rectal, subcutaneous or local routes of administration to treat the target site. The method of delivery chosen depends, among other things, upon the condition being treated, desired therapeutic concentration of the medicament and the duration of medicament concentration that must be maintained at the target site.

Recently, there has been considerable interest in delivering medicaments via the oral cavity (e.g., gums, buccal, and sublingual areas, etc.). Delivery to target sites of the oral cavity has several advantages. One advantage is that it allows localized treatment of the teeth, gums and other soft tissues. Another advantage is that the oral cavity has an extensive network of blood capillaries under the mucosa that is particularly suited to provide rapid and effective systemic absorption of systemic medicaments.

The oral cavity contains a variety of cell types arranged in many layers and structures that make it an interesting and unique site for drug delivery. Transmucosal drug delivery aims to introduce systemic agents through the oral mucosa. Mucosal drug delivery is directed to delivery of drugs locally, rather than systemically.

Oral tissues have different physical and chemical absorptive properties. For example, the teeth and bones of the oral cavity are hard tissues and the alveolar mucosa, gingiva, tongue, palate, lips, cheeks and the floor of the mouth are soft tissues, all of which possess different properties; therefore, they require different formulations to maximize drug delivery to them. Additionally, there are multiple desired effects within the same tissue, which means that the physical properties of formulations and other drug delivery materials may need to change in order to maximize the desired effect.

This disclosure seeks to address the physical and chemical properties required of various oral drug delivery formulations, which when used in known quantitative and qualitative parameters, would maximize the delivery of a drug formulation for its intended purpose.

Currently, there are oral patches, rinses, topical applications, sponges, irrigation devices, oral appliance designs, and other oral appliances which have not utilized various principles of material science to advance the application of the medicaments to the many types of oral tissues present in the oral cavity.

There is therefore a clinical need for an easy-to-use oral appliance that can be used at home to treat various diseases of the oral cavity. In order to optimize drug delivery and therefore clinical efficacy this easy-to-use oral appliance takes into consideration different material science properties of the many types of tissues found in the oral cavity.

### SUMMARY

By modifying the material science properties of the oral appliance, enhanced medicament delivery can be obtained. The oral appliances provided are configured to enhance medicament delivery. This is in contrast to conventional oral appliances that are standard and medicament is added to those standard oral appliances without considering modifying the material science properties of the oral appliance to enhance medicament delivery. The material science properties of the oral appliance include the modification of the oral appliance by using materials, such as for examples, ceramics, metals, polymers, or combination thereof to obtain properties to enhance medicament delivery. These properties include, among other things, the hardness, softness, elasticity, flexibility, pressure exerted on the tissue when the oral appliance is worn. Other properties of materials used in the oral appliance include density, strength, hydrophobicity, hydrophilicity, charge, and/or porosity. In some embodiments, the oral appliances are enhanced to allow for optimal patient comfort and therefore enhanced patient compliance.

The oral appliance has an interior surface which is configured to contour a treatment area and deliver a medicament thereto, the treatment area comprising soft tissue, hard tissue or a combination of soft tissue and hard tissue, at least a portion of the interior surface of the oral appliance being about as soft or as hard or softer or harder to match the softness or hardness of the treatment area. Conversely, the interior surface can be harder than or softer than the apposition tissue it is applying medicaments to.

In one aspect, there is an oral appliance for delivering a medicament to an oral cavity, the oral appliance having an interior surface and an exterior surface, the interior surface and/or exterior surface configured to contour a treatment area and deliver medicament thereto, the treatment area comprising soft tissue, hard tissue or a combination of soft tissue and hard tissue, at least a portion of the interior surface and/or exterior surface of the oral appliance being less, equal or greater in hardness, elasticity, flexibility, density, porosity, hydrophobicity, hydrophilicity relative to the treatment area.

In one embodiment, there is an oral appliance for delivering a medicament to an oral cavity, the oral appliance having an interior surface and an exterior surface, the interior surface and/or exterior surface configured to contour a treatment area and deliver medicament thereto, the treatment area comprising soft tissue, hard tissue or a combination of soft tissue and hard tissue, at least a portion of the interior surface and/or exterior surface of the oral appliance being about as soft or as hard to match the softness or hardness of the treatment area.

In other aspects, an oral appliance is provided for delivering a medicament, having an exterior and an interior, the interior configured to contour at least a portion of the teeth and/or soft tissue areas of the oral cavity, the interior, exterior or both the interior or exterior of the oral appliance comprising a plurality of microneedles, the microneedles configured for delivering the medicament to the oral cavity.

In certain aspects, an oral appliance for delivering a medicament to an oral cavity is provided, wherein the oral appliance includes an exterior and an interior, the interior of the oral appliance configured to contour at least a portion of the teeth and/or soft tissue areas of the oral cavity, the interior, exterior or both the interior and exterior of the oral appliance comprising a polymer and a ceramic material, metal material, composite or a combination thereof, the interior, exterior or both the interior and exterior of the oral appliance containing the medicament for delivering the medicament to the oral cavity.

In various embodiments, an oral appliance for delivering a medicament to an oral cavity is provided, wherein the oral appliance has an exterior and an interior, the interior of the oral appliance configured to contour at least a portion of the teeth and/or soft tissue areas of the oral cavity, the interior, exterior or both the interior and exterior of the oral appliance comprising the medicament and a biodegradable polymer configured to degrade and deliver the medicament to the oral cavity.

In many embodiments, a method of making an oral appliance for delivering a medicament to an oral cavity is provided. The method includes providing an oral appliance having an interior surface, the interior surface configured to contour a treatment area and deliver medicament thereto, the treatment area comprising soft tissue, hard tissue or a combination of soft tissue and hard tissue, at least a portion of the interior surface of the oral appliance being about as soft or as hard to match the softness or hardness of the treatment area; and dispensing the medicament in the interior surface of the oral appliance for delivery to the oral cavity.

A method of treating a diseased tissue of an oral cavity is provided. In some embodiments, the diseased tissue can be a wound. The method of treatment includes providing an oral appliance for delivering a medicament to an oral cavity, the oral appliance having an interior surface, the interior surface configured to contour a treatment area and deliver medicament thereto, the treatment area comprising soft tissue, hard tissue or a combination of soft tissue and hard tissue, at least a portion of the interior surface of the oral appliance being about as soft or as hard to match the softness or hardness of the treatment area, the interior surface having a medicament disposed at discrete regions of the interior surface for delivering the medicament to the diseased tissue.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE FIGURES

In part, other aspects, features, benefits and advantages of the embodiments will be apparent with regard to the following description, appended claims and accompanying drawings.

FIG. 1A illustrates an enlarged side view of an embodiment of the oral appliance configured to cover the teeth or hard tissues and/or soft tissues of a patient, the oral appliance shown without teeth and/or soft tissues inserted in the oral appliance.

FIG. 1B illustrates an enlarged side view of an embodiment of the oral appliance configured to cover the teeth or hard tissues and/or soft tissues of a patient, the oral appliance shown without teeth and/or soft tissues inserted in the oral appliance, however, the oral appliance contains metal, ceramic material, polymer material, and medicament on the interior and exterior surfaces at discrete regions.

FIG. 1C illustrates an enlarged side view of an embodiment of the oral appliance configured to cover the teeth or hard tissues and/or soft tissues of a patient, the oral appliance shown without teeth and/or soft tissues inserted in the oral appliance, however, the oral appliance contains biodegradable material and medicament on the interior and exterior surfaces at discrete regions.

FIG. 1D illustrates an enlarged top view of an embodiment of the oral appliance configured to cover the teeth or hard tissues and/or soft tissues of a patient, the oral appliance shown without teeth and/or soft tissues inserted in the oral appliance, however, the oral appliance contains memory shape material on the interior and exterior surfaces.

FIG. 1E illustrates an enlarged side view of an embodiment of the oral appliance configured to cover the teeth or hard tissues and/or soft tissues of a patient, the oral appliance shown without teeth and/or soft tissues inserted in the oral appliance, however, the oral appliance contains a plurality of microneedles on the interior and exterior surfaces.

FIG. 1F illustrates an enlarged side view of an embodiment of a plurality of microneedles.

FIG. 1G illustrates an enlarged side view of another embodiment of a plurality of microneedles.

FIG. 2A illustrates an enlarged view of the regions along the sulcus (e.g., gumline) where the medicament of the oral appliance will be loaded in a polymeric matrix (e.g., hydrogel).

FIG. 2B illustrates an enlarged interior view of an image of the oral appliance that is made by taking the base image of the oral cavity and creating a digital image that additively layers over the oral cavity including the teeth, gums, soft tissue areas and/or the palate.

FIG. 3 illustrates an enlarged cross-sectional view of the anatomy of the gums and a tooth including free gingiva, attached gingiva, lining mucosa, the periodontal pocket or crevice, the cementoenamel junction (CEJ), periodontal ligament, cementum, the enamel, dentin, pulp and the junctional epithelium. In some embodiments, the periodontal pocket is targeted for delivery. The design of the medicament composition of the oral appliance is to target the periodontal pocket or crevice of the sulcus and extrude the medicament into the entrance of the periodontal pocket.

FIG. 4 illustrates an enlarged cross-sectional view of a portion of the oral appliance. In the embodiment shown, medicament is disposed in a porous material that is a hydrogel at a discrete region of the oral appliance. The hydrogel is shown in an uncompressed state and when worn with slight pressure, the hydrogel will be compressed against, among other things, the gingival crevice or periodontal pocket causing a seal or encapsulation of the entrance of the gingival crevice or periodontal pocket, which prevents oral fluids (e.g., saliva, exudate or other captured fluids upon insertion of the device, etc.) from entering the crevice or pocket, which allows release of the medicament in the gingival crevice or periodontal pocket and allows the hydrogel to absorb or wick fluid from the crevice or pocket.

FIG. 5 illustrates an enlarged cross-sectional view of a portion of the oral appliance that is placed adjacent to the teeth and gums. In the embodiment shown, medicament is disposed in a porous material that is a hydrogel at a discrete region of the oral appliance. The hydrogel is shown in a compressed state, where the device is worn and the hydrogel is compressed against, among other things, the gingival crevice or periodontal pocket causing a seal or encapsulation of the entrance of the gingival crevice or periodontal pocket, which limits or may even prevent oral fluids (e.g., saliva, gingival crevicular fluid (GCF), exudate, or other captured fluids upon insertion of the device, etc.) from entering the crevice or pocket. The hydrogel allows release of the medicament into the gingival crevice or periodontal pocket to treat the inflamed tissue shown by the down arrows. The hydrogel also absorbs or wicks oral fluids from the crevice or pocket, which aides healing, shown by the up arrows.

It is to be understood that the figures are not drawn to scale. Further, the relationship between objects in a figure may not be to scale and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter can at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a medicament” includes one, two, three or more medicaments.

The term “porous” as used herein, refers to a material which is permeable such that fluids are movable therethrough by way of pores or other passages. An example of a porous material is a hydrogel material, a cellulosic material, concrete, ceramics, foams, sponges and derivatives thereof. The porous material may be the result of using a low or high molecular weight polymer. In some embodiments, the polymer may be porous as it is printed at a low density on the oral appliance and/or substrate, or is printed in a geometric pattern, either as a specific structure or a randomized structure. In some embodiments, all or a portion of the oral appliance before the hydrogel is loaded in the device may be porous.

The term “non-porous” as used herein, refers to a material which is impermeable such that fluids cannot move through the material. The non-porous material may be the result of using a low or high molecular weight polymer. In some embodiments, the polymer may be non-porous as it is applied at a high density on the oral appliance and/or substrate in a solid form with no structural spacing to hold medicament. In some embodiments, all or a portion may be non-porous before the hydrogel is loaded in the device.

The term “hydrogel” or “hydrogels” refer to a broad class of polymeric materials, that may be natural or synthetic, which have an affinity for an aqueous medium (e.g., an antimicrobial), and are able to absorb amounts of the aqueous medium, but which do not normally dissolve in the aqueous medium.

The term “medicament” as used herein, is generally meant to refer to any substance that alters the physiology of a patient. The term “medicament” may be used interchangeably herein with the term “medicine”, “medication”, “drug”, “therapeutic agent”, “therapeutically effective amount”, or “active pharmaceutical ingredient”. It will be understood that a “medicament formulation” may include more than one therapeutic agent, wherein exemplary combinations of therapeutic agents include a combination of two or more medicaments. The medicament can also include cells, where the device (e.g., oral appliance) can be seeded with the cells, for example, gingival cells or gingival tissue, bone cells, cartilage cells, bone tissue so that the device can repair or replace tissue in the treatment area.

The term “antimicrobial” as used herein is generally refers to a group of medicaments that reduce, inhibit, or eliminate microbial growth. An antimicrobial includes an antibiotic, an antifungal, an antiprotozoal, an antiviral or a combination thereof. The term “antimicrobial” may be used interchangeably herein with the term “antimicrobial agent.” It will be understood that an “antimicrobial formulation” may include more than one antimicrobial agent, wherein exemplary combinations of antimicrobial agents include a combination of two or more antimicrobials.

The term “dental plaque” is a general term for the diverse microbial community (predominantly bacteria) found on the tooth surface, embedded in a matrix of polymers of bacterial and salivary origin.

The term “oral diseases” refers to diseases, wounds and disorders affecting the oral cavity or associated medical conditions. Oral diseases include, but are not limited to, inflammation, infection, dental caries, periodontal diseases (e.g., gingivitis, adult periodontitis, early-onset periodontitis, chronic periodontitis and/or aggressive periodontitis) or the like. Inflammatory diseases can also include benign and malignant tumors such as Lichen Planus and squamous cell carcinoma, respectively, as well as various yeast and fungal infections and conditions like Xerostomia.

The term “gingiva” or “gum” refers to a dense fibrous tissue and overlying mucous membrane enveloping alveolar processes of upper and lower jaws and surrounding the necks of teeth.

The term “gingivitis” refers to inflammation of gingival tissue without loss of connective tissue.

The term “inflamed tissue” refers to bone, teeth, or gingival tissue, that is red, swollen and can be painful. It will also more broadly include dental caries and/or hypersensitive areas of the teeth. Inflammation can be caused by trauma to the oral cavity, infection or other causes.

The term “periodontal disease” refers to an inflammatory process of the gingival tissues and/or periodontal membrane of the teeth, resulting in a deep gingival sulcus, possibly producing periodontal pockets and loss of alveolar bone.

The term “periodontitis” refers to inflammation and loss of connective tissue of the supporting or surrounding structure of teeth with loss of attachment.

The terms, “treating” or “treatment” includes “preventing” or “prevention” of disease. In addition, “treating” or “treatment” does not require complete alleviation of signs or symptoms, does not require a cure, and specifically includes protocols that have only a marginal effect on the patient.

The term “localized” delivery includes delivery where one or more medicaments contact the tooth and/or soft tissue areas, for example, the gingival margins of the teeth or a region inside of the mouth such as the palate, or in close proximity thereto.

The term “targeted delivery” includes delivery of one or more medicaments at the target site as needed for treatment of the disease or condition including cosmetic applications, for example, whitening teeth or removing stains. In some embodiments, the oral appliance can be used to deliver antimicrobial to the soft tissue of the inside of the mouth including, but not limited to, any soft tissue adjacent or between the teeth including, but not limited to, the papilla, tissue of the upper and lower dental arches, marginal gingiva, gingival sulcus, inter-dental gingiva, gingival gum structure on lingual and buccal surfaces up to and including the muco-gingival junction and/or the palate and/or the floor of the mouth. In various embodiments, the soft tissue area includes the muco-buccal folds, hard and soft palates, the tongue, lining mucosa, and/or attached gingival tissue.

The term “custom fit” as used herein, refers to an oral appliance that is specifically made via molding and/or 3D printing, to correspond to at least a portion of a tooth, a selected number of teeth, all of the teeth and/or soft tissues found in the mouth of a specific individual patient. A custom fit oral appliance is not a generic device which is then heated or otherwise manipulated by a consumer, inserted into their mouth by themselves and then molded by that consumer to fit their own mouth. The patient image is the result of an action upon that particular individual by another person whereas the consumer is acting upon himself/herself by manually manipulating the generic material.

In some embodiments, custom fit includes situations where the patient images himself or herself with a scanning device including those in a computer or smart devices (e.g., smartphones such as Apple iPhones, Android devices, Samsung Galaxy devices, or the like, as well as tablets such as Apple iPad, Microsoft Surface, or the like) and then the appliance is made as a separate act.

The term composite includes is a material made from two or more constituent materials with different physical or chemical properties that, when combined, produce a material with characteristics different from the individual components. Composites include, for example, a polymer combined with a metal, a polymer combined with a ceramic material, a polymer combined with a ceramic material and a metal.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

The headings below are not meant to limit the disclosure in any way; embodiments under any one heading may be used in conjunction with embodiments under any other heading.

### Oral Appliance

By modifying the material science properties of the oral appliance, enhanced medicament delivery can be obtained. The material science properties of the oral appliance include the modification of the oral appliance by using materials, such as for examples, ceramics, metals, polymers, or combination thereof to obtain properties to enhance medicament delivery. These properties include, among other things, the hardness, softness, elasticity, flexibility, pressure exerted on the tissue when the oral appliance is worn, density, strength, hydrophobicity, hydrophilicity, charge, and/or porosity of the oral appliance.

The oral appliances provided are configured to enhance medicament delivery. This is in contrast to conventional oral appliances that are standard and medicament is added to those standard oral appliances without considering modifying the oral appliance to enhance medicament delivery. In some embodiments, the oral appliances provided allow comfort to the patient to enhance patient compliance.

In some embodiments, there is provided an oral appliance for delivering a medicament to an oral cavity. The oral appliance has an interior surface and/or exterior surface configured to contour a treatment area and deliver a medicament to it. The treatment area comprises soft tissue, hard tissue or a combination of soft tissue and hard tissue, at least a portion of the interior surface and/or exterior surface of the oral appliance being about as soft or as hard to match the softness or hardness of the treatment area. In other aspects, the oral appliance comprises an exterior surface and all or a portion of the interior surface and/or exterior surface contains a medicament, the interior surface and/or exterior surface having a softness to match the treatment surface it contours. In yet other aspects, the oral appliance comprises an exterior surface and all or a portion of the interior surface and/or exterior surface contains a medicament, the interior surface and/or exterior surface having a hardness to match the treatment surface it contours. In many aspects, the interior surface and/or exterior surface of the oral appliance comprises a polymeric matrix disposed at discrete regions inside the interior surface or on the exterior surface of the oral appliance. The hard tissue of the oral cavity comprises at least a tooth and/or other bony structures of the oral cavity. The soft tissue of the oral cavity comprises muco-buccal folds, soft palate, hard palate, lining mucosa, buccal and labial mucosa, attached and unattached gingival tissue, and floor of the mouth tissue. The polymeric matrix can include a polymeric material, for example a hydrogel, which can be soft to match the soft tissues of the oral cavity or it can be hard to match the hard tissues of the oral cavity or it can vary between those two parameters. In some instances, the oral cavity also includes artificial surfaces. Many artificial surfaces can be hard surfaces, for example, those created by a dental bridge or a dental crown.

Numerous custom fit oral appliances can be made in a variety of ways including by traditional thermoforming, 3D printing or additive manufacturing, or injection molding or other ways. Unlike orthodontic appliances, the present oral appliance is not designed to move teeth and is not an orthodontic appliance. Therefore, a plurality of oral appliances will be configured to fit the teeth in the same position as was imaged within the oral cavity. The teeth position will not change. However, the medicament disposed in or on the oral appliance will be in the same or different areas at different stages of the treatment regimen with a variety of oral appliances. Thus, kits containing a plurality of oral appliances can be provided with different treatment plans. For example, as the patient condition improves, each oral appliance may have a decreasing amount of antimicrobial or the medicament can change as the treatment progresses.

In various embodiments, the oral appliance is monolithic or a single piece and the interior surface custom fit and formed to fit contours of the teeth and/or soft tissue areas inside the oral cavity of a patient in need of treatment. In some aspects, the medicament is disposed at discrete regions in the interior surface of the oral appliance. In other aspects, the medicament can be disposed in a channel located in the interior surface of the oral appliance. In certain embodiments, the oral appliance comprises, consists essentially of or consists of one, two, three, four, five or more oral appliances.

In various embodiments, the oral appliance is not monolithic or a single piece. The medicament is disposed in a polymeric matrix disposed at discrete regions or in a continuous channel inside the oral appliance, as a separate component of the oral appliance. For example, the medicament can be disposed in a polymeric matrix which contains a porous material (e.g., hydrogel) that is configured to allow release of the medicament when the oral appliance is worn.

In some embodiments, oral appliances include, but are not limited to, oral trays, oral holders, oral covers, or the like that are designed to be placed within the oral cavity. The interior surface and/or, in some aspects, the exterior surface of the oral appliance contains a medicament disposed inside the porous portion of the polymeric matrix of the oral appliance.

Numerous different oral appliances can be made by the methods of the present application, including custom fit oral appliances that correspond to a digital scan taken from the patient's mouth or impression molds. Custom fit oral appliances are generally described in U.S. Pat. No. 6,626,669 filed Apr. 15, 2002; U.S. Pat. No. 9,579,178 filed Jul. 12, 2013 and U.S. Pat. No. 9,649,182, to Peter J. Zegarelli, filed Jun. 18, 2015. The entire disclosure of this patent is herein incorporated by reference into the present disclosure.

The oral appliance when worn allows the interior and/or exterior surface of the oral appliance to be adjacent to the teeth and/or gums or other tissue in the oral cavity. In some embodiments, the oral appliance receives one or more teeth including one or more molars, premolars, incisors, cuspids, tooth implant, or combinations or portions thereof.

In various embodiments, the non-porous material is the structural backbone of the oral appliance and is present throughout the oral appliance to give it form, shape and structural integrity. The porous material parts (e.g., hydrogel) of the oral appliance are strategically placed about the oral appliance in order to deliver medicaments to those areas to be treated. These areas can be either internal or external to the oral appliance.

In some embodiments, the oral appliance can be made by controlling the print density of the polymer during manufacturing (e.g., 3D printing, injection molding, etc.). For example, the same polymer can be used at a density of, for example, 0.25 g/cm³ to 0.5 g/cm³ at discrete regions to form the porous regions of the oral appliance and at a higher density for example, 0.8 g/cm³ to 1.5 g/cm³ to make the oral appliance non-porous at discrete regions.

In some embodiments, the oral appliance can be made by controlling the density of the polymer during 3D printing or additive manufacturing. For example, different polymers can be printed using two or more print heads, each print head having a different polymer. A high-density polymer can be used (e.g., 50,000 MW) and printed at discrete regions to form the non-porous regions of the oral appliance and another print head can use a low-density polymer (e.g., 5,000 MW) to make the oral appliance porous at discrete regions.

It will be understood that the oral appliance with discrete portions of the porous material and with discrete portions of non-porous material can be monolithic or a single piece having the same or different material. This type of oral appliance, in some embodiments, does not contain a porous insert after the oral appliance is made. Such porous inserts are described in U.S. Pat. No. 9,579,178, filed Jul. 12, 2013 to Peter J. Zegarelli. The entire disclosure of this patent is herein incorporated by reference into the present disclosure.

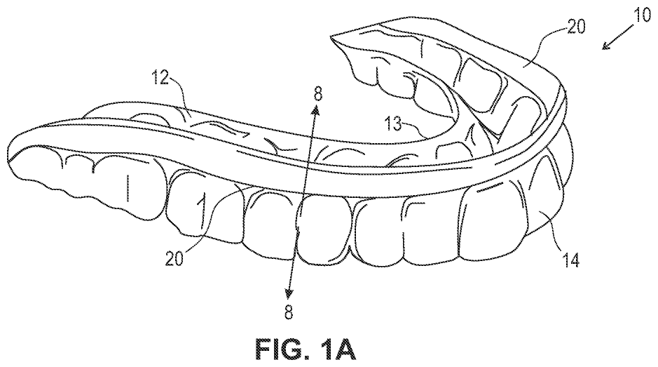

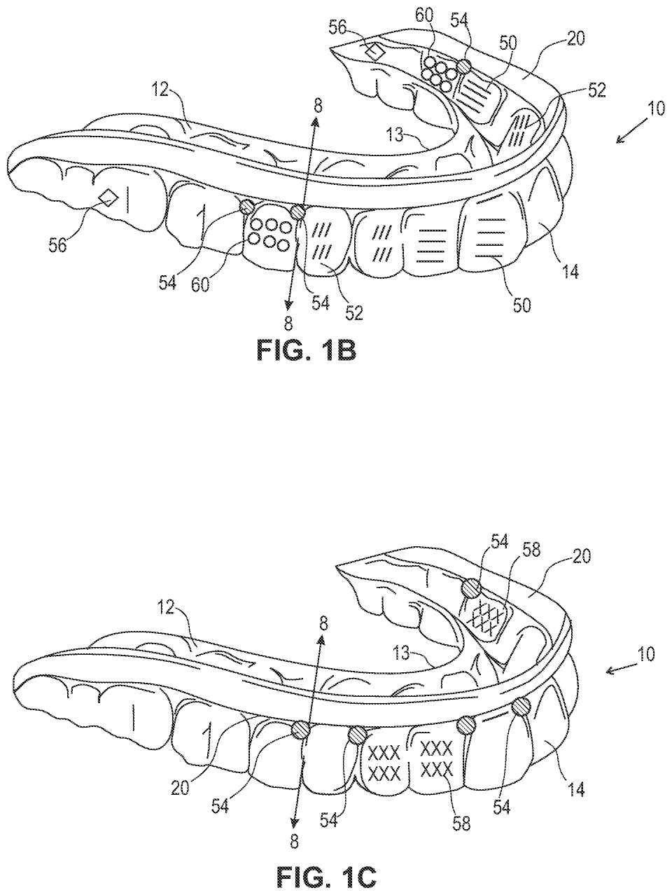

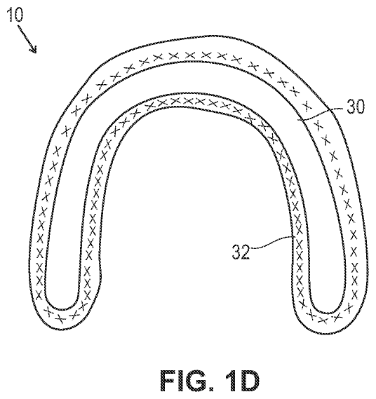

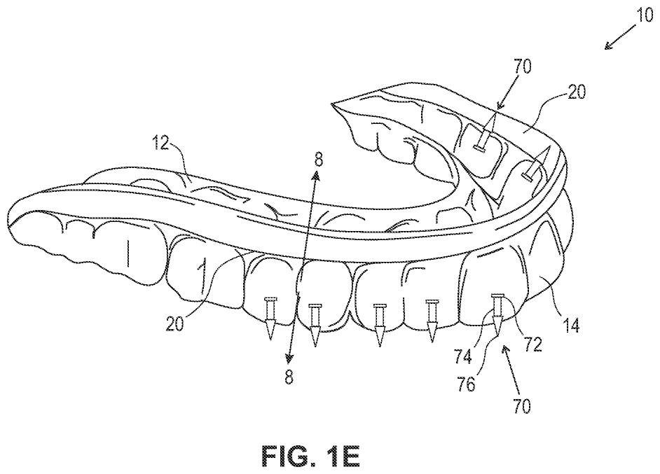

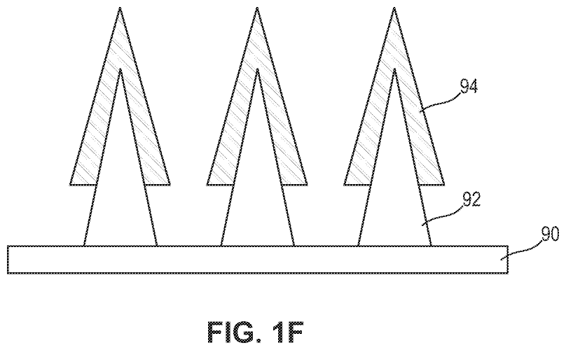

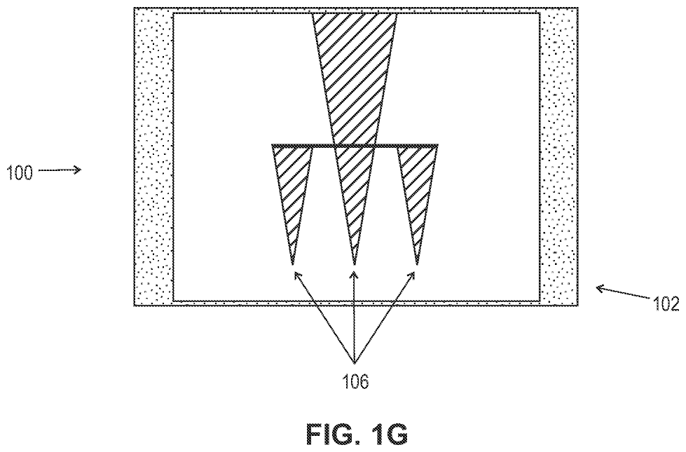

FIG. 1A is an enlarged side view of an embodiment of the oral appliance 10. The oral appliance has a shell or scaffold 13 to receive and/or hold medicament and/or oral tissue. The oral appliance has an interior surface 12 and exterior surface 14, both comprising, in some embodiments, a polymer. The interior surface 12 is configured to contact one or more teeth and/or soft tissue areas of a patient. The interior surface 12 is custom fit to the individual patient's mouth and configured to contour at least a portion of teeth and/or soft tissues of the oral cavity. In this view the interior surface 12 is configured to contact the teeth and soft tissue. Oral appliances include, but are not limited to, oral trays, oral holders, oral covers, or the like that are designed to be placed within the oral cavity. The interior surface 12 of the oral appliance contains a medicament disposed in a polymeric matrix (not shown) and the medicament can be disposed anywhere within the oral appliance. For example, the medicament can be disposed at discrete regions adjacent to the treatment area or uniformly disposed throughout the device. As the interior and/or exterior surface of the oral appliance contacts the oral cavity, the medicament is released from the polymeric matrix (e.g., hydrogel) by all or parts of the oral appliance contacting the desired treatment site or pressure from the device contacting tissue or fluid at the treatment site (e.g., gums, tissue, teeth, etc.).

In some embodiments, the polymeric matrix containing the medicament can degrade over time by the action of enzymes, by hydrolytic action and/or by other similar mechanisms in the oral cavity. In some embodiments, all or discrete portions of the polymeric matrix containing a medicament will degrade and release the medicament at or near the target site in the oral cavity. The oral appliance will cover at least a portion of the teeth and or gums by applying the device over axis 8-8 to cover the area of the teeth and or gums, and the oral appliance will be adjacent to the gingival sulcus 20, which will allow the medicament, if desired, to be released from the polymer to this area.

FIG. 1B is another embodiment of oral appliance 10, wherein the oral appliance comprises an exterior or exterior surface 14, and an interior or interior surface 12. The oral appliance has shell or scaffold 13 to receive and/or hold medicament and/or oral tissue. The interior surface 12 is configured to contour at least a portion of teeth and/or soft tissue areas of the oral cavity. The interior, exterior or both the interior or exterior of the oral appliance 10 comprises a polymer material 50 and a ceramic material 52, a metal material 56, a composite 60 or a combination thereof, the interior, exterior or both the interior or exterior of the oral appliance containing the medicament for delivering the medicament 54 to the oral cavity. It will be understood that the entire device can be made of a polymer material, a ceramic material, a metal material, or a composite. Alternatively, the device can be made from a polymer material, a ceramic material, a metal material, or a composite material disposed at discrete regions of the oral appliance. Alternatively, the device can be made from a polymer material and have disposed a ceramic material, a metal material, and/or a combination thereof in the polymer throughout the entire oral appliance or at discrete regions of the oral appliance. The medicament can be disposed at those discrete regions.

FIG. 1C is another embodiment of oral appliance 10, wherein the oral appliance comprises an exterior or exterior surface 14 and an interior or interior surface 12. The oral appliance has shell or scaffold 13 to receive and/or hold medicament and/or oral tissue. The interior of the oral appliance is configured to contour at least a portion of teeth and/or soft tissue areas of the oral cavity, the interior, exterior or both the interior or exterior of the oral appliance comprise a medicament 54 and a biodegradable polymer 58 configured to degrade and deliver the medicament to the oral cavity. It will be understood that the entire device can be made of a biodegradable polymer material. Alternatively, the device can have the biodegradable material disposed at discrete regions of the oral appliance. The medicament can be disposed at those discrete regions.

FIG. 1D is yet another embodiment of oral appliance 10, wherein the oral appliance comprises an exterior or exterior surface and an interior or interior surface. The interior of the oral appliance is configured to contour at least a portion of the teeth and/or soft tissue areas of the oral cavity, the interior, exterior or both the interior and exterior of the oral appliance comprise a memory shape material 32 configured to engage, directly or indirectly (where, there will be material disposed between the oral cavity tissue and the memory shape material), at least a portion of the teeth and/or soft tissue areas of the oral cavity and the interior, exterior or both the interior and exterior of the oral appliance comprising a medicament for delivery to the oral cavity. FIG. 1D also illustrates a channel 30 disposed in the interior surface of oral appliance 10. The channel can be configured to receive medicament and/or teeth. In this embodiment, the oral appliance is made with memory shape material. The memory shape material can be polymeric material that has the ability to return from a temporary shape to its original or permanent shape on application of an external stimulus. For example, the memory shape material can expand to a temporary shape when the oral appliance is worn about at least a portion of the teeth and/or soft tissue areas of the oral cavity. When the oral appliance is removed, the memory shape material can contract to its original shape. In this way, treatment can be comfortably and discretely directed to the treatment area of the oral cavity. In the embodiment, shown in FIG. 1D, the memory shape material is in the oral appliance and has a horseshoe shaped configuration and borders the teeth.

FIG. 1E is another embodiment of oral appliance 10, oral appliance having an exterior or exterior surface 14 and an interior or interior surface 12, the interior configured to contour at least a portion of teeth and/or soft tissue areas of the oral cavity, the interior, exterior or both the interior or exterior of the oral appliance comprising a plurality of microneedles 70, the microneedles configured for delivering the medicament to the oral cavity. Each microneedle has a base portion 72, a tip end portion 76 distal to the base portion and a body 74 therebetween.

FIGS. 1F and 1G are different embodiments of a plurality of microneedles useful for the oral appliance of this disclosure. FIG. 1F shows a plurality of coated microneedles, each having a body portion 92, a coating portion 94 containing medicament and a base portion 90. FIG. 1G shows a plurality of microneedles 106 attached to a reservoir 104 containing a medicament and disposed on the exterior 102 of oral appliance 100.

In use, the microneedles can be coated with medicament at their tip and the wearer can move soft tissue in the oral cavity (e.g., a tongue and/or inner lip) to allow the microneedles to pierce the soft tissue and deliver medicament or alternatively release the medicament. For example, the microneedle can be coated with nitroglycerine 0.2 mg or 0.4 mg, or other dosage. When the wearer has chest pain, the wearer can move his or her tongue by the microneedle and the microneedle can release the medicament locally, the blood flow to the oral cavity is enough to allow the nitroglycerine to be absorbed orally to dilate the heart blood vessels and relieve the chest pain.

In some embodiments, the microneedles have a reservoir to hold medicament and when pressure is applied to the oral appliance, the reservoir can release medicament in the area by exposing the microneedles.

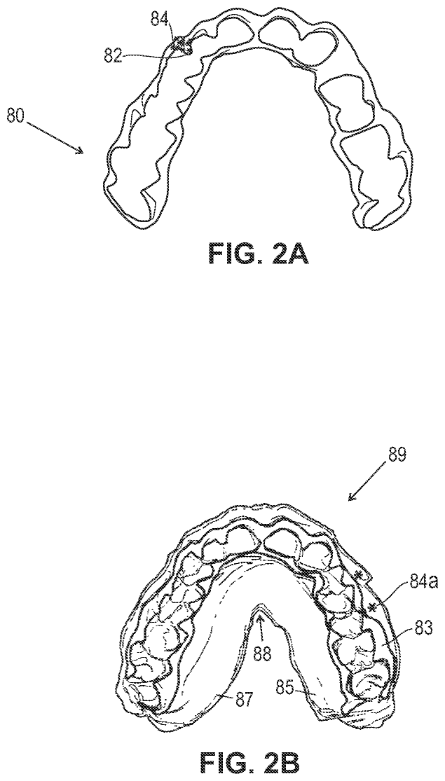

FIG. 2A illustrates an enlarged view of the regions along the sulcus (gumline) where the medicament composition 84 of the oral appliance will be loaded in a polymeric matrix (e.g., hydrogel) 80 and placed at discrete regions 82 inside the oral appliance.

FIG. 2B illustrates an enlarged view of the oral appliance 89 that has regions along the sulcus (gumline) 83 where the medicament 84a (e.g., chlorhexidine) will be loaded at discrete regions on the interior surface of oral appliance 89. The lower portion of the oral appliance corresponds to and will contact portions of the tongue and hard palate 85 as well as the soft palate 87 along each side of the oral cavity. The lower portion 88 of the oral appliance corresponds to and will contact portions of the tongue and hard palate 85 as well as the soft palate 87.

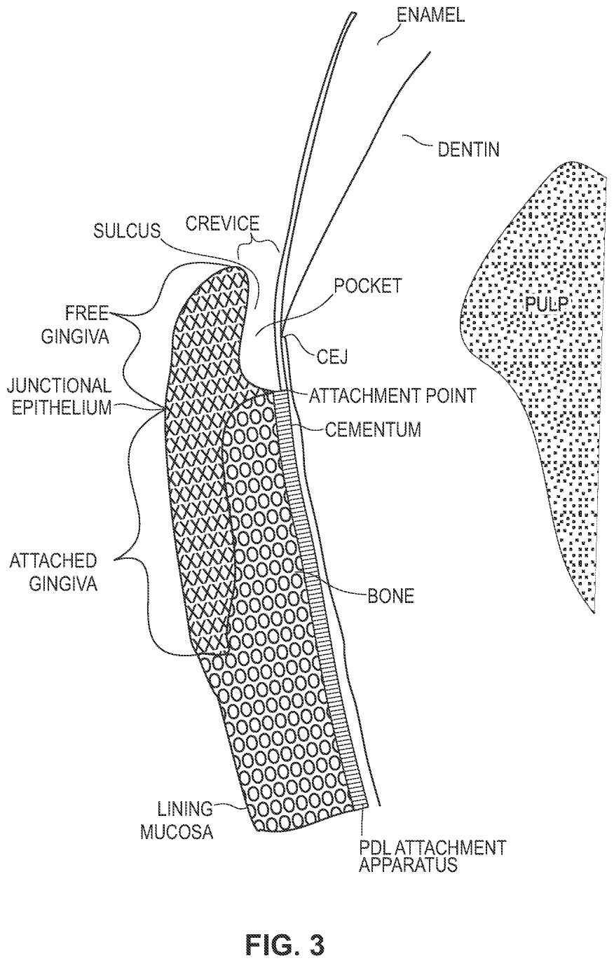

FIG. 3 illustrates an enlarged cross-sectional view of the anatomy of the gums and a tooth including free gingiva, attached gingiva, lining mucosa, the periodontal pocket or crevice, the cementoenamel junction (CEJ), periodontal ligament, cementum, the enamel, dentin, pulp and the junctional epithelium. In some embodiments, the treatment area is the periodontal pocket that is targeted for delivery.

In some embodiments, the oral appliance contains a polymeric matrix, which is, in many aspects, a porous material (e.g., a hydrogel), which can contain a medicament, for example, an antimicrobial (e.g., chlorhexidine), which is released for a sustained period of time until the infection is reduced and is brought under greater control. The oral appliance described herein serves multiple purposes. It holds the medicament in place with no or limited dilution by saliva or contamination by oral liquids, and it keeps the medicament at the top of the pocket. As the hydrogel is squeezed when the oral appliance is worn, the medicament diffuses into the top of the pocket. The oral appliance design, characterized by the hydrogel placement over the gingival crevice, is akin to an encapsulation device, sealing off the opening of the pocket from outside contamination by saliva and other liquids, and forcing the hydrogel containing medicament into the pocket entrance.

By encapsulating the gingival crevice, which is the entrance to the periodontal pocket, the oral appliance assures that the captured fluids, which are those fluids coating and surrounding the teeth and soft tissues when the tray is inserted, are pushed away from the crevice entrance and kept away by the encapsulating hydrogel over the crevice. Further, as the hydrogel is emptied of medicament, gingival crevicular fluids (GCFs) are wicked up and absorbed by the hydrogel in a fluid exchange, removing this contaminated exudate from the infected periodontium. GCFs include exudate fluids containing bacteria, dead cellular structures, interstitial fluids, inflammatory factors, etc. The greater the degree of inflammation due to the periodontal disease, the greater the rate of flow of the GCF. Removing the GCF from the pocket creates space for the medicament and/or hydrogel to occupy. This will result in decreased inflammation and thus decreased GCF flow. As the gingiva at the top of the pocket begins to respond to the medicament (e.g., the antimicrobial chlorhexidine), the surface inflammation will decrease, and the pocket will also shrink in depth. Once the antimicrobial treatment regimen has adequately reduced the pathologic microbiome, other avenues of treatment can be initiated to combat the other aspects of the periodontal disease sequence.

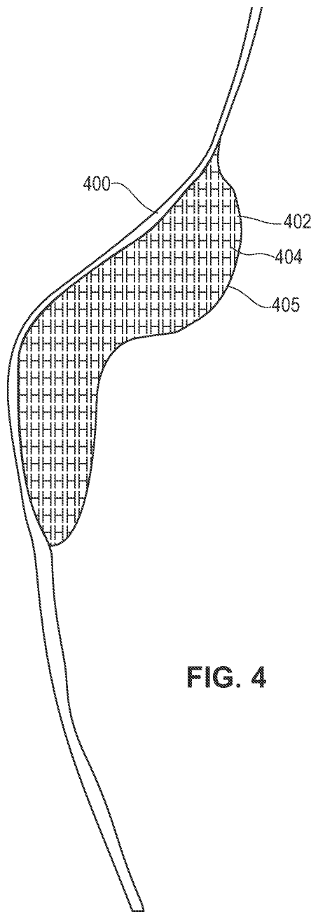

FIG. 4 illustrates an enlarged cross-sectional view of a portion of the oral appliance 400. In the embodiment shown, a medicament is disposed in a polymeric matrix which can be porous material 402. In some aspects, porous material 402 is a hydrogel 404 disposed at a discrete region of the oral appliance 404. The hydrogel is shown in an uncompressed state 405 and when worn with slight pressure, the hydrogel will be compressed against, among other things, the gingival crevice or periodontal pocket causing a seal of the entrance of the gingival crevice or periodontal pocket, which prevents other oral fluids (e.g., saliva, foreign liquids, exudate) from entering the crevice or pocket, which allows release of the medicament in the gingival crevice or periodontal pocket and allows the hydrogel to absorb or wick fluid from the crevice or pocket. In this embodiment, the hydrogel is disposed at a discrete region of the oral appliance and is sized to be greater than the height, width, and length of the entrance of the periodontal pocket or crevice. In this way, when the device is worn, the hydrogel will contact the entrance of the periodontal pocket or crevice and encapsulate and seal it. In some embodiments, when the device is worn there will be a gap formed between the entrance of the periodontal pocket or crevice (top portion) and the bottom of the pocket by the junctional epithelium that will allow, among other things, the medicament to leach out of the hydrogel and treat the top of the periodontal pocket.

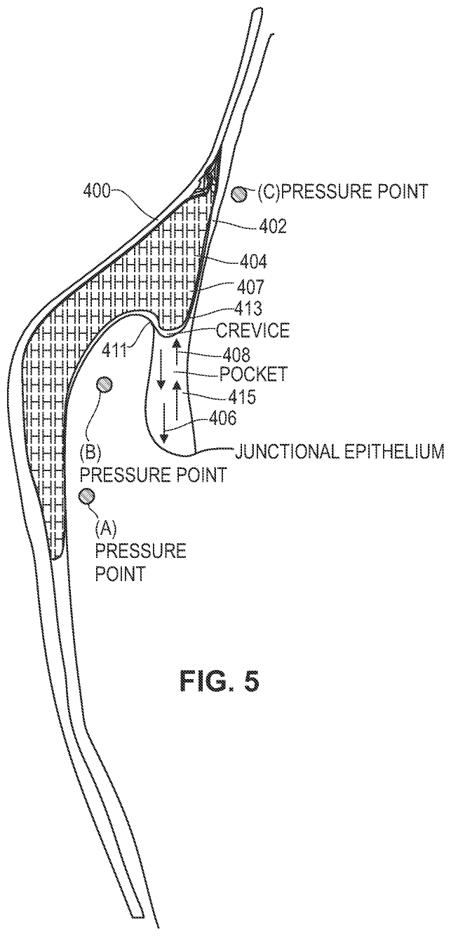

FIG. 5 illustrates an enlarged cross-sectional view of a portion of the oral appliance 400 being worn that is placed adjacent to the teeth and gums using the top down approach to treating periodontal disease. In the embodiment shown, a medicament is disposed in a polymeric matrix that is porous material 402, in this aspect, a hydrogel 404 at a discrete region of the oral appliance. The hydrogel is shown in a compressed state 407, where the device is worn and the hydrogel is compressed against, among other things, the gingival crevice or periodontal pocket causing a seal 413 of the entrance of the gingival crevice or periodontal pocket, which prevents oral fluids (e.g., saliva, foreign liquids, exudate, etc.) from entering the crevice or pocket. There is a gap 415 between the junctional epithelium and the entrance 411 of the crevice or pocket, which is now sealed by the hydrogel. This gap allows the hydrogel to release medicament in the gingival crevice or periodontal pocket to treat deep down into the inflamed tissue. The medicament release is shown by the down arrows 406. The hydrogel also absorbs or wicks oral fluids from the crevice or pocket which aides healing, shown by the up arrows 408. The hydrogel is placed in the oral appliance and is configured to create a seal at the entrance of the periodontal pocket so that there will be a bulge of hydrogel at the entrance to cause seal 413.

As the medicament is leached out of the hydrogel, empty hydrogel spaces open up and become available to absorb and remove crevicular/sulcular fluids from the environment. In this way, the hydrogel has dual ability to deliver the medicament and wicking action to remove crevicular/sulcular fluids from the environment. This dual action of wicking which then creates a negative crevicular fluid flow, allows the medicament under pressure, shown by pressure points A, B and C, to enter the top portion of the pocket to fill the resultant negative pressure void, thus inserting the medicament further into the pockets. Over sustained daily treatment regimens, the inflammation at the top of the pocket decreases and with decreased inflammation there is decreased swelling and therefore decreased pocket depth.

In an embodiment, the oral appliance described in this disclosure contains a polymeric matrix (e.g., hydrogel) disposed at discrete regions inside the interior surface of the oral appliance, wherein the medicament is configured to contact at least a portion of an inflamed tissue of the oral cavity including at least a portion of the subgingival space when the oral appliance is worn to cause delivery of the medicament to at least the portion of the subgingival space of the inflamed tissue. In another embodiment, the hydrogel seals an entrance of a gingival crevice or periodontal pocket of a portion of an inflamed tissue of the oral cavity as a portion of the hydrogel exudes from the oral appliance and contacts the entrance of the gingival crevice or periodontal pocket to reduce or prevent contamination of the gingival crevice or periodontal pocket by oral fluids.

### Oral Appliance Materials

The oral appliance can be made of any materials that can hold and release the medicament. The oral appliance has material science properties that complements and enhances the release of medicament depending on the condition that is being prevented or treated. The oral appliance can be made from porous material, non-porous material or a combination thereof. In some embodiments, the oral appliance is non-porous, however, porous material can be disposed at discrete regions of the oral appliance.

In various embodiments, the material from which the oral appliance can be made from includes swellable polymers, such as for example, hydrogels, gels, polymer brushes or combinations thereof.

In some embodiments, suitable polymers for use to make the oral appliance include, for example, polyacrylates, polyamide-imide, phenolic, nylon, nitrile resins, petroleum resins, fluoropolymers, copolyvidones (copovidones), epoxy, melamine-formaldehyde, diallyl phthalate, acetal, coumarone-indene, acrylics, acrylonitrile-butadiene-styrene, alkyds, cellulosics, polybutylene, polycarbonate, polycaprolactones, polyethylene, polyimides, polyphenylene oxide, polypropylene, polystyrene, polyurethanes, polyvinyl acetates, polyvinyl chloride, poly(vinyl alcohol-co ethylene), styrene acrylonitrile, sulfone polymers, saturated or unsaturated polyesters or combinations thereof.

In some embodiments, the polymer comprises, consists essentially of or consists of an amount from about 5% to about 100% by weight, from about 5%, 10%, 15%, 20%, 25%, 30%, 35%, 40%, 45%, 50%, 55%, 60%, 65%, 70%, 75%, 80%, 85%, 90%, 95% to about 100% by weight, from about 10% to about 15% by weight, from about 15% to about 20% by weight, from about 20% to about 25% by weight, from about 25% to about 30% by weight, from about 30% to about 35% by weight, from about 35% to about 40% by weight, from about 40% to about 45% by weight, from about 45% to about 50% by weight, from about 50% to about 55% by weight, from about 55% to about 60% by weight, from about 60% to about 65% by weight, from about 65% to about 70% by weight, from about 70% to about 75% by weight, from about 75% to about 80% by weight, from about 80% to about 85% by weight, from about 85% to about 90% by weight, from about 90% to about 95% by weight, or from about 95% to about 100% by weight of the oral appliance. In some embodiments, the oral appliance is substantially all polymer from about 80% to about 99.9% by weight. The medicament comprises, consists essentially of or consists of an amount from about 0.01% to about 50%, from about 0.1% to about 20% by weight, from about 0.5% to about 10%, or from about 1% to about 7% by weight of the oral appliance.

In various embodiments, the molecular weight of the polymer can be a wide range of values. The average molecular weight of the polymer can be from about 1,000 to about 10,000,000 g/mol; or about 1,000 to about 1,000,000; or about 5,000 to about 500,000; or about 10,000 to about 100,000; or about 20,000 to about 50,000 g/mol.

In some embodiments, when the oral appliance is made from one polymer, the density of the polymer can vary such that the non-porous and porous regions are formed in the oral appliance from a single material.

In some embodiments, when different molecular weight polymers are used, the polymer can be dense and have a higher molecular weight such that the polymer is non-porous. In some embodiments, the polymer can be less dense and have a lower molecular weight such that the polymer is porous. In some embodiments, the oral appliance can be made from multiple polymers, as described above. The multiple polymers can have the same or different densities. The polymers can have an average molecular weight of from about 1,000 to about 10,000,000 g/mol; or about 1,000 to about 1,000,000; or about 5,000 to about 500,000; or about 10,000 to about 100,000; or about 20,000 to about 50,000 g/mol.

In some embodiments, the oral appliance can have a modulus of elasticity (Young's modulus) in the range of about 1×10⁻² to about 6×10⁵ dynes/cm², or 2×10⁴ to about 5×10⁵ dynes/cm², or 5×10⁴ to about 5×10⁵ dynes/cm².

The polymer of the oral appliance may optionally have a viscosity enhancing agent such as, for example, hydroxypropyl cellulose, hydroxypropyl methylcellulose, hydroxyethyl methylcellulose, carboxymethylcellulose and salts thereof, Carbopol, poly-(hydroxyethylmethacrylate) (pHEMA), poly-(methoxyethylmethacrylate), poly(methoxyethoxyethyl methacrylate), polymethylmethacrylate (PMMA), methylmethacrylate (MMA), gelatin, polyvinyl alcohols, propylene glycol, mPEG, PEG 200, PEG 300, PEG 400, PEG 500, PEG 600, PEG 700, PEG 800, PEG 900, PEG 1000, PEG 1450, PEG 3350, PEG 4500, PEG 8000 or combinations thereof.

In some embodiments, the polymer can comprise a hydrogel that is or is not infused with at least one medicament. Suitable hydrogels for use in the oral appliance, include natural hydrogels, such as for example, gelatin, collagen, silk, elastin, fibrin and polysaccharide-derived polymers like agarose, and chitosan, glucomannan gel, hyaluronic acid, polysaccharides, such as cross-linked carboxyl-containing polysaccharides, or a combination thereof. Synthetic hydrogels include, but are not limited to those formed from polyvinyl alcohol, acrylamides such as polyacrylic acid and poly(acrylonitrile-acrylic acid), polyurethanes, polyethylene glycol (for example, PEG 3350, PEG 4500, PEG 8000), silicone, polyolefins such as polyisobutylene and polyisoprene, copolymers of silicone and polyurethane, neoprene, nitrile, vulcanized rubber, poly(N-vinyl-2-pyrolidone), acrylates such as poly(2-hydroxy ethyl methacrylate) and copolymers of acrylates with N-vinyl pyrolidone, N-vinyl lactams, polyacrylonitrile or combinations thereof.

In some embodiments, cross-linking agents used to make the porous material non-porous include, but are not limited to, 1-hydroxycyclohexyl phenyl ketone, glutaraldehyde, formaldehyde, epoxy, compounds, dialdehyde, sodium borate/boric acid, glyoxal, oxidized dextrins, epichlorohydrin, endogen polyamine spermidine, oxidized alginate, zinc, borax, ethylene glycol dimethacrylate (EGDMA), N, N′-methylenebisacrylamide, derivatives of ethylene glycol di(meth)acrylate, derivatives of methylenebisacrylamide, formaldehyde-free crosslinking agent including N-(1-Hydroxy-2,2-dimethoxyethyl)acrylamide, or a combination thereof.

In some embodiments, the oral appliance can be transparent so that a user can see the teeth. In other embodiments, the oral appliance can be translucent or opaque. The oral appliance may be disposable and sterilizable. In various embodiments, one or more components of the oral appliance is sterilized by radiation in a terminal sterilization step in the final packaging. Terminal sterilization of a product provides greater assurance of sterility than from processes such as an aseptic process, which require individual product components to be sterilized separately and the final package assembled in a sterile environment. Other methods may also be used to sterilize one or more components of the oral appliance, including, but not limited to, E-beam radiation, gamma radiation, gas sterilization, such as, for example, with ethylene oxide or steam sterilization.

In various embodiments, an oral appliance for delivering a medicament to an oral cavity is provided. The oral appliance comprises an exterior and an interior, the interior of the oral appliance configured to contour at least a portion of teeth and/or soft tissue areas of the oral cavity, the interior, exterior or both the interior or exterior of the oral appliance comprising a polymer material and a ceramic material, metal material or a combination thereof, the interior, exterior or both the interior or exterior of the oral appliance containing the medicament for delivering the medicament to the oral cavity. In some aspects, the polymer material comprises polyacrylates, polyamide-imide, phenolic, nylon, nitrile resins, petroleum resins, fluoropolymers, copolyvidones (copovidones), epoxy, melamine-formaldehyde, diallyl phthalate, acetal, coumarone-indene, acrylics, acrylonitrile-butadiene-styrene, alkyds, cellulosics, polybutylene, polycarbonate, polycaprolactones, polyethylene, polyimides, polyphenylene oxide, polypropylene, polystyrene, polyurethanes, polyvinyl acetates, polyvinyl chloride, poly(vinyl alcohol-co ethylene), styrene acrylonitrile, sulfone polymers, saturated or unsaturated polyesters or combinations thereof. In other aspects, the ceramic material comprises tricalcium silicate, dicalcium silicate, lithium disilicate or porcelain.

In many embodiments the oral appliance can include metal material, the metal material comprising alloys based on gold, silver, platinum, palladium, nickel, cobalt, iron, copper, titanium, tin, aluminum oxide, zinc oxide, or boron trinitride. In other aspects, the oral appliance can include a composite material which comprises silver-mercury amalgam, bisphenol A-glycidyl methacrylate (Bis-GMA), triethylene glycol dimethacrylate (TEGDMA), urethane dimethacrylate (UDMA) or 1,6-Hexanediol dimethacrylate (HDDMA).

In various embodiments, the oral appliance for delivering a medicament to an oral cavity includes an exterior and an interior, the interior of the oral appliance configured to contour at least a portion of teeth and/or soft tissue areas of the oral cavity, the interior, exterior or both the interior or exterior of the oral appliance comprising the medicament and a biodegradable polymer configured to degrade and deliver the medicament to the oral cavity. In some embodiments, the biodegradable polymer comprises polylactic acid, chitosan, polyglycolic acid or poly(lactide-co-glycolide) (PLGA), polylactide (PLA), polyglycolide (PGA), D-lactide, D,L-lactide, L-lactide, D,L-lactide-co-ε-caprolactone, L-lactide-co-ε-caprolactone, D,L-lactide-co-glycolide-co-ϵ-caprolactone, poly(D,L-lactide-co-caprolactone), poly(L-lactide-co-caprolactone), poly(D-lactide-co-caprolactone), poly(D,L-lactide), poly(D-lactide), poly(L-lactide), poly(esteramide) or a combination thereof.

In other embodiments, the oral appliance for delivering a medicament to an oral cavity comprises an exterior and an interior, the interior of the oral appliance configured to contour at least a portion of teeth and/or soft tissue areas of the oral cavity, the interior, exterior or both the interior or exterior of the oral appliance comprising a memory shape material configured to engage at least a portion of teeth and/or soft tissue areas of the oral cavity and the interior, exterior or both the interior or exterior of the oral appliance comprising a medicament for delivery to the oral cavity. In some aspects, the memory shape material comprises a shape memory polymer including polyphosphazenes, poly(vinyl alcohols), polyamides, polyester amides, poly(amino acid)s, polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyesters, polylactides, polyglycolides, polysiloxanes, polyurethanes, polyethers, polyether amides, polyether esters, polystyrene, polypropylene, polyvinyl phenol, polyvinylpyrrolidone, chlorinated polybutylene, poly(octadecyl vinyl ether) ethylene vinyl acetate, polyethylene, poly(ethylene oxide)-poly(ethylene terephthalate), polyethylene/nylon (graft copolymer), polycaprolactones-polyamide (block copolymer), poly(caprolactone) dimethacrylate-n-butyl acrylate, poly(norbornyl-polyhedral oligomeric silsesquioxane), polyvinyl chloride, urethane/butadiene copolymers, polyurethane block copolymers, styrene-butadiene-styrene block copolymers, and combinations thereof. In other aspects, the memory shape material comprises a thermoplastic blend shape memory polymer characterized by a Tg exceeding room temperature whose rubber modulus and elasticity are derived substantially from physical crosslinks wherein the shape memory polymer comprises a blend of a crystalline polymer selected from the group consisting of poly(vinylidene fluoride), polyglycolides, polylactide and copolymers thereof, poly(hydroxybutyrate), poly(ethylene glycol), polyethylene, polyethylene-co-vinyl acetate, poly(vinyl chloride), poly(vinylidene chloride) and copolymers of poly vinylidene chloride and poly vinyl chloride with an amorphous polymer selected from the group consisting of poly(vinyl acetate), poly methyl acrylate, poly ethyl acrylate, atactic poly methyl methacrylate, isotactic poly methyl methacrylate and syndiotactic poly methyl methacrylate. In yet other embodiments, the memory shape material comprises a memory shape alloy comprising TiNi, CuZnAl, or FeNiAl alloys.

### Material Science Properties of the Oral Appliance

The physical and chemical properties of the material used for the oral appliance can be selected to optimize the delivery of the medicament to a selected treatment area. Moreover, the application of material science properties of the oral appliance to oral mucosa has broad implications. For example, different geometries of the oral appliance can enhance the therapeutic effect of the structure on the oral cavity.

An oral appliance is in contact with multiple hard and soft tissues of the oral cavity. The oral appliance will have various material properties that affect its form and function. These material properties will influence the target tissue in physical ways by applying various degrees of targeted force and pressure to tissue, and in chemical ways by controlling the diffusion of drug onto the tissues of the oral cavity.

Different oral appliance materials can have various intensive material science properties, including without limitation, different stress-strain behavior with varying degrees of elasticity; strength measures; viscoelasticity; viscosity; plasticity; hardness; density; porosity; and permeability to water. The oral appliance will have structural (extensive) properties such as stiffness (or its complementary concept flexibility). These material science properties stem from the combination of several primary types of materials, namely metals, ceramics, polymers, and composites. Thus, oral appliances having a desirable combination of material science properties can be designed by using different types of materials.

Generally, oral appliances are comprised of and contain polymers. An oral appliance has two main components, namely a scaffold or backing which holds the medicament that will be delivered at a target tissue site, and the medicament. In this disclosure, the scaffold or backing is defined by an interior surface and an exterior surface of the oral appliance. The medicament can be formulated with a polymeric matrix which can deliver a drug via: (1) diffusion control, (2) matrix swelling, (3) matrix degradation, or (4) pendant group polymeric cleavage.

In various embodiments, there is provided an oral appliance for delivering a medicament to an oral cavity. The oral appliance has an interior surface and/or exterior surface configured to contour a treatment area and deliver medicament to it. The treatment area comprises soft tissue, hard tissue or a combination of soft tissue and hard tissue, at least a portion of the interior surface and/or exterior surface of the oral appliance being about as soft or as hard to match the softness or hardness of the treatment area. In other aspects, the oral appliance comprises an exterior surface and all or a portion of the interior surface and/or exterior surface contains a medicament, the interior surface and/or exterior surface having a softness to match the treatment surface it contours. In yet other aspects, the oral appliance comprises an exterior surface and all or a portion of the interior surface contains a medicament, the interior surface having a hardness to match the treatment surface it contours. In many aspects, the interior surface of the oral appliance comprises a polymeric matrix disposed at discrete regions inside the interior surface. The hard tissue of the oral cavity comprises at least a tooth and/or other bony structures of the oral cavity. The soft tissue of the oral cavity comprises muco-buccal folds, soft palate, hard palate, lining mucosa buccal and labial mucosa, attached and unattached gingival tissue, tongue and floor of the mouth tissue. The polymeric matrix can include a polymeric material, for example a hydrogel, which can be soft to match the soft tissues of the oral cavity or it can be hard to match the hard tissues of the oral cavity or it can vary to be softer or harder. In some instances, the oral cavity also includes artificial surfaces. Many artificial surfaces can be hard surfaces, for example, those created by a dental bridge or a dental crown.

When the polymeric matrix delivers the medicament by diffusion control, then the diffusion of medicament out of polymeric matrix depends on both pressure and concentration gradients. The pressure is controlled by the scaffold or backing of the oral appliance and the geometry of the polymeric matrix, and the concentration is controlled by polymeric matrix formulation, medicament viscosity, as well as the target tissue and fluidic environment. For drug delivery by matrix degradation, the oral appliance contains biodegradable polymers. Biodegradable polymeric matrices will degrade via surface or bulk degradation, depending on their geometry (e.g., structural property), rate of water penetration, and rate of polymeric cleavage (e.g., material property).

The polymeric matrix may also be producing a desired outcome in and of itself, without a drug, by applying pressure to tissue and/or by being made with an antimicrobial material, such as antiseptic material (e.g., silver ions, copper, and organosilane nanocoating). The oral appliance in its entirety can also be made of antiseptic material. This application is relevant to wound healing in the oral cavity such as with oral mucositis or dry mouth syndrome.

The oral appliance backing, scaffold or shell can support a porous polymeric matrix, which in various aspects can be hydrogel. The hydrogel can enhance the degree of apposition to the tissues and enhance the degree of the desired compression of the porous material. This compression will affect drug delivery by creating a pressure gradient on the medicament. The hydrogel acts as a holding vehicle to store the medicament at discrete regions into or onto the oral appliance and then dispense it at targeted areas of the oral cavity. The backing, scaffold or shell of the oral appliance is defined by the interior and exterior surface of the oral appliance and holds the hydrogel, in some embodiments, in the interior surface of the oral appliance. In some embodiments, the shell of the oral appliance does not affect the oral tissues. In some embodiments, the porous material (e.g., hydrogel) does not affect the oral tissues since it has no support. Together the shell and the porous material having medicament disposed at discrete regions of the oral appliance are designed to complement each other using material science properties to enhance delivery of the medicament. Additionally, the material science approach to designing a complete system comprising the shell and the porous material (e.g., hydrogel) also considers the comfort of the appliance for the patient who wears it. Comfort is important for patient compliance in any treatment modality and the characteristics such as smoothness, ease of insertion and removal, comfort when inserted and others can all be influenced by the material properties used.

Teeth are very hard and do not compress. Tooth bleaches are caustic and can burn the soft tissues surrounding each tooth and cause sensitivity to the exposed roots of teeth. Common plastic oral appliances which hold a liquid or hydrogel cannot stop these liquids from leaking onto these sensitive areas upon insertion. However, a custom fit oral appliance containing a properly formulated hydrogel can minimize these adverse effects. A hydrogel which is designed to appose the enamel portions of the teeth and not touch the roots or gums is firm with little to no compressibility. A firm or hard hydrogel can be lightly supported by the oral appliance backing with little compression by the oral appliance material and can keep the bleach on the tooth and prevent it from extravasating away from the enamel. Also, the firmer hydrogel can be formulated to release the bleach slowly so as to enhance the bleaching efficacy. In this case, the material properties of the shell can be soft since a hard shell would cause more compression and flatten the hydrogel onto soft tissues, which are susceptible to burning by the bleach.

Caries can occur on any surface of a tooth, but certain surfaces are more susceptible than others. Occlusal surfaces with all the grooves and fissures, the softer roots, interproximal (e.g., between teeth) areas are examples of such surfaces. In this situation, the hydrogel can be sponge-like and easily compressible to enhance the release of the anti-caries agents that can flow over the entire surface of the tooth freely, even in areas difficult to access. In this situation, the properties of the oral appliance shell can be those which enhance the compression of the hydrogel to maximize the dispersal of the medicament. In some embodiments, the oral appliance can be firm to enhance compression of the hydrogel and to maximize the dispersal of the medicament. In bleaching and caries prevention the same hard tissue, teeth, is being treated, however the different needs of each application affects the material properties of the hydrogel and the oral appliance tray in order to enhance the delivery of the medicament in each case.

In other embodiments, when the oral appliance is used to treat gingivitis, there are hard tissues (e.g., teeth) and soft tissues (e.g., gingiva and mucosa) that need to be considered, but the point of treatment is the gingiva and gingival margin. This is the case because gingivitis is characterized by inflammation of the gingiva. Since the gums are inflamed and swollen, in this application the hydrogel would be formulated to be softer and spongier to adequately bathe the gums and to continue to contact these soft tissues as the gums heal and recede from a swollen state. In this case, the oral appliance shell chosen can have physical properties which not only support the hydrogel but also seal tightly against the hard tissues in order to keep the medication on the gums. For example, in some embodiments, the oral appliance can be firm and not only support the hydrogel but also seal tightly against the hard tissues in order to keep the medication on the gums.

In another embodiment, when the oral appliance is used to treat periodontal disease, in order to facilitate healing of the chronic wound associated with periodontal disease, there are hard tissues and soft tissues to be considered with the point of attack for treatment being the gingival margin and the crevice/sulcus below it. The fluid dynamics of gingival crevicular fluid (GCF), which emanate out of the periodontal pocket, impeding the projection of medications into the pocket can also be considered. In this situation, the hydrogel needs to be formulated so as to cap off the crevice from outside fluids and overwhelm the outward GCF so as to project the medicament from the top of the pocket, down into the depths of the pocket and hold them there. Here the bolus release of the medicament enhanced by the proper hydrogel formulation will serve two purposes. The first is the medicament release and the second is the absorption of the GCF in to the now emptied hydrogel. These competing fluids will flow past each other from their respective areas of high concentration to the areas of low concentration until they reach equilibrium after several minutes. The absorption of the GCF in the hydrogel will serve to rid the pockets of these exudates once the oral appliance is disposed of and discarded. But the medicament will remain. The goal is to gradually, over time and with repetitive treatments, further project the medicament deeper and deeper into the pockets. As healing occurs, the GCF flow will decrease due to decreased inflammation. Here also, the oral appliance shell material sciences can be formulated in order to complement the dual actions of the hydrogel and a semi-firm shell would be considered while a firmer hydrogel is used not to bathe the gingiva as in gingivitis but to cap off the crevice and have a more sustained release of medications while wicking up the GCFs.

In another embodiment, in terms of treating dry mouth (xerostomia) or oral mucositis where the oral tissues are generally desiccated and may or may not have ulcerations of the soft tissues, an oral appliance is needed to gently treat the tissues and hydrate them and medicate them over long periods of time. Here the hydrogel needs to be formulated to slowly release the medicament over long periods of time such as during the sleeping hours. The interior and exterior surfaces of the device may be utilized to hydrate partially or all of the tissues of the oral cavity. The oral tissues here, especially the non-keratinized mucosal tissues of the buccal mucosa, labial mucosa, floor of the mouth and the lateral borders of the tongue, are very friable and require an especially smooth surface, which will not substantially adhere to the tissues and pull the epithelium off creating more ulcerations. Here the shell of the oral appliance may need to be very soft and flexible and the hydrogel should be likewise formulated. The hydrogel should have a release profile of medicament that sufficiently hydrates and treats the conditions present with Biotene® and Biotene like formulations (e.g., formulations containing, for example, the enzymes mutanase, dextranase, lysozyme, lactoperoxidase, and glucose oxidase), topical steroids for the ulcerations and anti-fungal medications for any infections present. These may be used singly or in combination with each other or other medications useful for these purposes. In some embodiments, the oral appliance can have the medicament glycerin or other moisturizing agent that can moisturize or lubricate tissues in the oral cavity and be used to treat or prevent, among other things, dry mouth.

The oral appliance shell and hydrogel for the applications discussed in this disclosure can have two functions, namely, to deliver medicament to the gumline and to seal the teeth from the medicament. In delivering the medicament to the gumline, the oral appliance material sciences can be altered such that the medicament can reach the inflamed area sub-gingivally and maintain an adequate concentration of medicament over the course of treatment. In sealing the teeth from the medicament, the material sciences should enhance close apposition of the shell to the teeth.

In various embodiments, along the gumline of the oral appliance there will be a channel in which the polymeric matrix, in some aspects a hydrogel, can be placed. The hydrogel can be dispensed, and UV cured in the oral appliance. In some embodiments, the hydrogel can be dispensed without a need for curing. The hydrogel can be adhered to the oral appliance and can release the medicament over time, for example, as part of a sustained release formulation. There are proprietary formulations of hydrogel, each having different material properties. In addition to material properties, the hydrogel can also have a drug release profile.

In various embodiments, maintaining the concentration of the medicament over the course of treatment depends upon the release profile of the medicament formulation. One factor that will determine a useful release profile is the ability of the oral appliance to seal the medicament to the gumline. In some embodiments, if the seal is flawed, the medicament can have more of a sustained release profile, so that if the medicament gets washed away it will be replenished continuously. If most of the medicament was delivered upfront with a compromised seal, it would be washed away, leaving less medicament at the target region. In other aspects, if the seal is good, more medicament can be immediately delivered, as the medicament would be localized, and the higher concentration would promote diffusion into the pocket. Often, since the seal is likely compromised and there is leakage into interproximal areas, the drug release profile will be both delivered based on both immediate and sustained release. In many aspects, the medicament of the oral appliance is delivered to the oral cavity as a sustained release dosage form or an immediate release dosage form or some combination thereof. In other embodiments, the medicament of the oral appliance is delivered to the oral cavity in a slow release dosage form.

In many aspects, in order to facilitate leaching of the medicament into the gingival sulcus or periodontal pocket, the diffusion, pressure gradient between the hydrogel and the subgingival space and capillary action are three parameters that can be affected by material science property adjustments between the shell and the porous material (e.g., hydrogel).

Each oral appliance has two pressure lines, one line on the gum and another line on the teeth. Between these two oral appliance pressure lines, against the gumline, there will be a layer of hydrogel impregnated with medicament. The pressure line of the oral appliance can apply pressure apically to the junctional epithelium and thus, cause the free gingiva to bend away from the tooth. The gingival attachment point effectively acts as a fulcrum point. This therefore allows for the medicament that is impregnated in the hydrogel to leach directly into the gingival sulcus or periodontal pocket.

In some aspects, the oral appliance is configured to apply apical pressure to a junctional epithelium as a portion of the hydrogel leaches into a gingival sulcus or a periodontal pocket. In other aspects, the medicament is released from the oral appliance into the gingival sulcus or periodontal pocket by enhancing pressure gradients, diffusion, and/or capillary action.

In some embodiments, the concentration gradient of medicament (higher concentration of the medicament in hydrogel relative to the subgingival space) can cause the medicament to diffuse into the subgingival space. The changing geometry of the subgingival space allows for an increased convection current of the hydrogel. In other embodiments, the increased volume of the subgingival space (e.g., gingival sulcus or periodontal pocket) caused by the apical pressure can decrease the pressure of the subgingival space, thereby creating a pressure gradient between the hydrogel and the subgingival space. This will cause fluid to flow from the hydrogel into the subgingival space. In yet other embodiments, the capillary action of the microfluidic environment on the crevice/sulcus/periodontal pocket can cause the medicament from the hydrogel to climb into the subgingival space.

In various aspects, by maintaining constant volume of the medicament at the gumline through material science property adjustments, the oral appliance will have the ability to minimize the gingival crevicular fluid flow, and thereby maintain adequate concentration of a medicament, e.g., chlorhexidine in the subgingival space. For example, studies with 2% chlorhexidine irrigation indicate that this antimicrobial rinse had to be applied 3 times in 10 minutes in order to maintain an adequate concentration, because it was being washed out by gingival crevicular fluid flow.

Constant volume of the chlorhexidine at the gumline can decrease gingival crevicular fluid flow by the following mechanism. Gingival crevicular fluid (GCF) originates as transudate from gingival capillaries. The crevicular fluid flow rate is governed by (i) the hydrostatic pressure gradient between gingival capillaries and the gingival sulcus; (ii) the osmotic pressure gradient between the gingival capillaries and the gingival sulcus; and (iii) the surface area of gingival capillaries. In healthy gums, GCF is classified as serum transudate. The lower hydrostatic pressure of the gingival sulcus relative to the tissue causes the fluid (e.g., mostly water) to flow across the capillary membranes of the gingival tissue into the gingival sulcus. The higher osmotic pressure of the tissue, however, causes less fluid to flow out of the tissue. These variables change in inflamed, diseased gums. In inflamed gums, vasodilation occurs, which increases the surface area of the capillaries at the sulcus, thereby increasing the crevicular fluid flow rate. In diseased gums, plaque-derived molecules accumulate in the subgingival space. This causes an increase in the osmotic pressure of the subgingival space, increasing the fluid flow rate from the gingiva to the pocket. The gingival crevicular fluid is classified as inflammatory exudate. Vasodilation allows inflammatory molecules and proteins to exude out with the fluid, changing the composition of GCF, allowing it, in some aspects, to become a diagnostic tool as more particularly described in U.S. Pat. No. 10,314,537 published on Jun. 11, 2019 and U.S. Ser. No. 16/392,195 filed on Apr. 23, 2019, both incorporated herein in their entirety.

The GCF in a diseased state consists of water, various cellular structures and their contents, inflammatory factors, exudate and other substances. Here the material science properties of the hydrogel can be adjusted to have hydrogel spaces large enough to wick up, hold and store in the appliance such that after use it is disposed of removing these diseased contents from the periodontium.

By virtue of the seal of the oral appliance, the crevicular fluid will not be able to flow out of the hydrogel/subgingival space. There will be constant volume at the gumline. Thus, the crevicular fluid flow will occur only until the point of equilibrium of hydrostatic pressure is reached, when the subgingival hydrostatic pressure equals the gingival capillary hydrostatic pressure. The hydrostatic pressure of the subgingival space will be constant. This equilibrium hydrostatic pressure prevents more transudate/exudate from being produced across the capillary membranes. Over time, the concentration of chlorhexidine will reach equilibrium between the hydrogel and the subgingival space, allowing the concentration of chlorhexidine in the subgingival space to be held relatively constant, that is without being washed out of the space by flowing gingival crevicular fluid. Thus, combined, the oral appliance can deliver a medicament (e.g., chlorhexidine) sub-gingivally and maintain concentration, including minimum inhibitory concentration (MIC) or minimum bactericidal concentration (MBC), while sealing it off from the teeth, avoiding staining and other adverse effects all enhanced by the manipulation of the material sciences of the hydrogel and the shell materials.

In many embodiments, generally, a custom-fit oral appliance fits around the patient's teeth tightly, however, interproximal areas may still not be tightly sealed. Nevertheless, the leakage of medicament on the anterior and posterior surfaces of the teeth can be minimized by utilizing materials for the oral appliance that possess elasticity, hardness and stiffness. In certain embodiments, the hydrogel of the polymeric matrix seals at least one surface of at least one tooth.

The above examples reference either the soft tissue gingiva or the hard tissue teeth and bone. These examples all concern the mucosal delivery of medicaments to the surface of these tissues. Gingiva is a firm keratinized tissue, which is attached to the underlying teeth and bone and is generally non-movable except the portion, the free gingiva and the unattached gingiva, which can be moved by an external force. There are no muscle attachments to gingiva, therefore, one cannot move it at will. For mucosal or transmucosal delivery of medicaments to the non-keratinized tissues of the mouth, for example the buccal and labial mucosa, the floor of the mouth, the ventral surface of the tongue (the dorsal surface is keratinized), the oral appliance can have its material science properties adjusted to take into account the very fragile nature of these tissues. Here, a small rough spot can easily lead to a cut and the resultant canker sore which can last for days and be very painful. Therefore, the shell can be very smooth and somewhat flexible in order to match not only the tender nature of these tissues but also the ability of these tissues to move at will due to the muscle attachments below the surface. Here, also the porous material (e.g., hydrogel) can be somewhat soft, but can have its chemistries adjusted to serve the requirements of drug delivery. For instance, with nitroglycerine where one needs an immediate bolus of drug delivery, the hydrogel can be formulated to release the drugs in that manner Conversely, with analgesics, the hydrogel can be formulated to release the drugs more slowly and sustained. Here again the material science properties affect how the drug is administered.

Elasticity of an oral appliance relates to the ability of the body of an oral appliance to resist a distorting influence and to return to its original size and shape when that influence or force is removed. When the teeth enter an oral appliance, they deform the material of the oral appliance, however, the material maintains the internal stress (or restoring force) to push back onto the teeth. If the material of the oral appliance changes its shape in response to forces (yields, or exhibits plastic behavior), then the oral appliance would not fit as tightly against the gums. The material will have a definitive stress-strain curve that will be used for quality control. A creep response curve and stress relaxation curve will also be used in quality control to ensure elasticity. The shape of the graph curve can provide reliable information, but a modulus of elasticity of the oral appliance will likely be sufficient.

Hardness of the material of an oral appliance relates to a measure of the resistance to localized plastic deformation induced by either mechanical indentation or abrasion. When the teeth enter the oral appliance, the teeth deform the elastic oral appliance locally, i.e., the oral appliance can be relatively soft so that there is no gap where fluid material can get through. The material of the oral appliance can have a definitive hardness as measured by a Shore durometer. Other durometers that can be used include those made by Rockwell, Vickers, or Brinell. The resultant hardness graph will be sufficient and will be used in quality control to ensure proper hardness. Together, the elasticity and hardness of the material of an oral appliance will allow the oral appliance to better form-fit or custom-fit around the teeth of a patient.

Another property of the material of the oral appliance is stiffness. The oral appliance can be relatively stiff. Three different but related properties hard/soft, rigid/elastic, and stiff/flexible are also properties of the material of an oral appliance. The modulus of elasticity of a material is not the same as the stiffness of a component made from that material. Modulus of elasticity is a property of the constituent material, while stiffness is a property of a structure or component of a structure, and hence it is dependent upon various physical dimensions that describe that component. That is, the modulus is an intensive property of the material while stiffness is an extensive property of the solid body that is dependent on the material and its shape and boundary conditions. Thus, when the oral appliance is in its final form, it will have a certain overall flexibility. Because of the horseshoe shape of the line of teeth, there will be a significant moment arm around the maxillary and mandibular central incisors, namely, the two front teeth. If the oral appliance is allowed to bend significantly around those teeth, the oral appliance will be hard to insert. Therefore, the oral appliance in its final form can have a definitive stiffness or inflexibility. This may mean adjusting the thickness of the oral appliance in certain areas or overall. In some embodiments, the oral appliance may be too hard and not sufficiently elastic and may not have the flexure properties sufficient for it to be able to go over the various curves of the teeth and boney undercut shapes of the jaws to allow it to fully seat in the oral cavity.

In many aspects, when making the oral appliance of this disclosure, various types of modulus of elasticity come under consideration. For example, the hard tissue of the oral cavity comprises at least a tooth and/or other bony structure of the oral cavity, while the soft tissue of the oral cavity comprises muco-buccal folds, soft palate, lining mucosa, buccal and labial mucosa, attached gingival tissue, tongue and mouth floor tissue. Thus, the hard tissue of the oral cavity has a modulus of elasticity of from about 1330 to about 1650 MPa, while the soft tissue of the oral cavity has a modulus of elasticity of from about 2.4 to about 19.8 MPa. In various aspects, the oral appliance has a modulus of elasticity from about 0.01 to about 19 GPa. The oral appliance of this disclosure contains a polymeric matrix disposed at discrete regions inside the interior surface of the oral appliance. The modulus of elasticity of the polymeric matrix comprises a hydrogel having a modulus of elasticity from about 8 to about 60 kPa. In some embodiments the hydrogel is considered soft and has a modulus of elasticity from about 8 to about 10 kPa. An example of soft hydrogel is 3% wt. polyethylene glycol dimethacrylate (PEGDMA) having a molecular weight (MW) of 1000 and 17% wt. PEGDMA having a molecular weight of 20,000, both crosslinked with methacrylate acid (MAA). On the other hand, 10% wt. PEGDMA MW 1000 and 10% wt. PEGDMA MW 20,000 crosslinked with MAA can provide a hard hydrogel having a modulus of elasticity from about 50 to about 60 kPa. In other embodiments, the hydrogel can be poly(vinyl alcohol) (PVA) and the oral appliance can be ethyl vinyl acetate (EVA).

In some embodiments, soft hydrogels can be used as polymeric matrix for a medicament which comprises an anticariogenic agent, an antimicrobial or a combination thereof. In other embodiments, a hard hydrogel can be used as a polymeric matrix for a medicament which comprises a bleaching agent, which when applied to a tooth's enamel, the bleaching agent does not exude from the tooth enamel.

In various embodiments, the oral appliance will apply a predetermined unit of pressure to the tooth side of the oral appliance, and a predetermined unit of pressure to the gum side of the oral appliance. Without the use of a ridge, the pressure of the oral appliance against the tooth and gums will seal the oral appliance to the tooth and the gum above and below the gum line. The oral appliance material can be slightly flexible. The gum side of the oral appliance will apply the pressure apically to the free junctional epithelium. The free junctional epithelium is the line that is formed where gingival tissue begins its attachment to the tooth. Free gingiva, the part of the gum that is not attached to the tooth, extends coronally to this point.

In certain embodiments, the shape of the free junctional epithelium can vary, depending on the patient's attachment levels. In the healthiest gums, the attachment of the gums to the teeth is at or near the cementoenamel junction (CEJ). However, the attachment throughout the mouth can be below that point. In diseased gums, attachment is often apical to the CEJ by a few millimeters. Wherever the attachment levels are, i.e., whatever shape the free junctional epithelium takes, the oral appliance will be custom fit apically to the free junctional epithelium.

### Structural Properties of the Oral Appliance

In various embodiments, the ability of the oral appliance to function as intended is influenced by its structural and geometric properties. There are several parameters to manipulate in designing the oral appliance. These parameters include height, thickness and shape. In many embodiments, the overall height of the oral appliance can differ between top and bottom oral appliances. Some oral appliances can fit tighter in certain areas than in others. In some embodiments, oral appliances can protrude by about 0.3 mm at the height of contour near the CEJ and/or by about 0.1 mm on the attached gingiva apical to the channel comprising the polymeric matrix or hydrogel. These protrusions will apply some pressure to enhance the seating of the oral appliance in the mouth.

The dimensions of the oral appliance, among other things, can depend on the target treatment site and whether local or systemic delivery of the medicament is required. The oral appliance can be adapted to any size and shape to receive at least a portion of the teeth and/or soft tissue areas inside the mouth to deliver the medicament. For example, the oral appliance is designed to contour, support and hold the polymer hydrogel material and, in various embodiments, extends to at least the muco-gingival junction, or at least 2 mm to 5 mm buccally or lingually beyond a gingival margin, or contacts all or substantially all of one or more teeth and/or soft tissue areas inside the mouth and adjacent buccal and lingual soft tissue areas, including the entire palate, the labial surfaces, the floor of the mouth and the tongue.

In certain embodiments, the thickness of the oral appliance can vary depending on whether the oral appliance was 3D printed or prepared by conventional manufacturing. In various embodiments, the oral appliance has a thickness of from about 0.06 inches to about 0.2 inches, a depth of at least about 1 mm to about 5 mm and a width of from about 1 mm to about 10 mm. In certain embodiments, the thickness of the surface of the oral appliance can be incrementally changed to be configured for treatment of a gum disease.

In various embodiments, the oral appliance has a thickness of from about 0.06 inches to about 0.2 inches. In some embodiments, the oral appliance can have a uniform thickness or a non-uniform thickness ranging from about 0.06, 0.07, 0.08, 0.09, 0.10, 0.11, 0.12, 0.13, 0.14, 0.15, 0.16, 0.17, 0.18, 0.19, to about 0.2 inches. The oral appliance can have a uniform or non-uniform thickness of about 0.2 to about 0.5 inches. In some embodiments, the oral appliance comprises a semi-solid construction.

In some embodiments, the dimensions of the polymeric matrix (e.g., gel, hydrogel, etc.), among other things, will depend on the target diagnosis site and whether local or systemic collection of the biological material is required as well as the type of biological material collection profile to achieve. In some embodiments, the oral appliance is prepared primarily of polymer material and can be adapted to any size and shape required to receive at least a portion of the teeth and/or soft tissue areas inside the mouth to collect the biological material. For example, the polymeric matrix may, in various embodiments, extend to at least the muco-gingival junction, or at least 2 mm to 5 mm buccally or lingually beyond a gingival margin, or contact all or substantially all of one or more teeth and/or soft tissue areas inside the mouth and adjacent buccal and lingual soft tissue areas. In various embodiments, the polymeric matrix contacts all or substantially all of one or more teeth and/or soft tissue areas inside the mouth. In various embodiments, the polymeric matrix contacts the soft tissue and teeth at or near a gingival margin or sulcus. In various embodiments, the polymeric matrix has a thickness of from about 0.06 inches to about 0.2 inches, a depth of at least about 1 mm to about 5 mm and a width of from about 1 mm to about 10 mm.

As discussed in this disclosure, in certain aspects, the polymeric matrix of the oral appliance is disposed in a channel in the interior surface of the oral appliance. In various embodiments, the polymeric matrix is hydrogel. In certain embodiments, the hydrogel can be recessed in the channel, be at the channel level, or be protruding from the channel. An example of a polymer matrix (e.g., hydrogel) is shown in FIG. 2A that can be inserted into the oral appliance.

In various embodiments, when the hydrogel is recessed in the channel, the hydrogel is placed more sparsely in the channel, such that it is sunk in the channel, and some space is allowed for the free gingiva to open, which could facilitate leaching of a drug into the periodontal pocket. The trade-off would be less pressure on the gumline, which can expedite healing. In certain embodiments, when the hydrogel is placed at the channel level, the free gingiva would be against a hard type of hydrogel whose properties might allow for some pressure while still allowing for some movement of the free gingiva. In other embodiments, the hydrogel protrudes from the channel. When the hydrogel protrudes from the channel, it can extend over and out of the channel, so that more pressure can be placed on the gumline, which could facilitate healing by the very pressure applied, while diffusing out the medicament.

In many aspects, the factors that could determine how much hydrogel could be dispensed into the channel include pressure at the gumline, which could facilitate healing and the movement of the free gingiva, which could facilitate leaching of medicament into the periodontal pocket.

In some aspects, when dispensing the hydrogel and loading it with chlorhexidine, for example, the hydrogel will swell. This can cause linear expansion of the hydrogel out of the channel, and that difference should be considered when dispensing the hydrogel. Generally, in an anisotropic swelling of a device, such as an oral appliance, an increase in the mass of water is equivalent to volume of expansion, namely the density of water or 1 g/cc. Since the expansion is in all three directions then the change (linear distance) in any one direction is the cube root of the swelling increase. For example, 58% swelling means a 1.58 swelling ratio (e.g., a 1-gram dry piece would weight 1.58 g after soaking). In this case the cube root of 1.58 is 1.16 indicating that each direction would increase by 16% for this swelling ratio (e.g., 2 mm length would swell to 2.32 mm length). As such the material properties of the shell and the hydrogel can be formulated to maximize the effect desired.

In various embodiments, the volume of medicament to be held in the polymeric matrix is another parameter for consideration when loading an oral appliance. For example, if the dry hydrogel has a density comparable to that of raw poly(HEMA) (1.15 g/ml), then, a 0.2 ml equivalent volume of hydrogel (based on the 2×4×26 mm for the oral appliance) would weigh 0.23 grams. If the dry hydrogel swells 58% (gains 58% of its weight in water) then 0.23×0.58=0.133 ml of aqueous solution that the hydrogel can hold. The concentration (mass % of medicament/volume water) of this solution multiplied by this volume would give the mass of the loaded medicament. In some embodiments, the medicament can be loaded in PVA and the oral appliance can be made of EVA polymeric matrix.

Generally, in an oral appliance, the hydrogel is disposed along the gumline in a channel There are several parameters to manipulate in designing the channel. In some aspects, the shape of the channel can be rounded and have a depth of about 1.5 mm and a height of about 3 mm, both dimensions to be customized depending on the patient. In other aspects, the depth of the channel can be form about 1.0 mm to about 3.5 mm and the height can vary from about 2 mm to about 6 mm. The width of the channel can vary depending on the application and can be continuous along the entire gumline or can be disposed at discrete regions to coincide to areas of the oral cavity to be treated.

In various embodiments, the height of the channel can be adjusted based on the dimension of the periodontal pocket. For example, if a patient has a periodontal pocket of about 6 mm and the channel is only 4 mm, the tray could be putting pressure on the bottom (apical) portion of the pocket and will not reach the top of the pocket. If the hydrogel is in line or protruding from the channel, the ability of the free gingiva to move and facilitate leaching is negligible and reaching the deep pocket may not be affected by that ability, or reaching the deep pocket is simply irrelevant. However, if the hydrogel is dispensed in the “sunk-in” fashion, the ability of the free gingiva to move and facilitate leaching becomes important. Thus, the channel can be deeper to allow the entire free gingiva (deep pocket to free gingival margin) to bend and facilitate leaching of medicament into pocket. An example of a channel is shown in FIG. 1D.

In other embodiments, drug delivery can occur transmucosally. The goal in transmucosal delivery is to deliver drugs systemically through the vascular soft tissues of the mouth. Drug uptake in some situations may need to be slow and sustained (e.g., statins, anti-cancer drugs) and in other situations it may need to be quick (e.g., nitroglycerin). In both situations, the characteristics of the oral appliance and the polymeric matrix or hydrogel can be modulated to fit the use by manipulating the material science properties of both substances. Using the material and structural properties outlined above, various drug release profiles can be attained. In addition, the use of microneedles and other micro or nano systems can be utilized in delivering drugs transmucosally. An example of an oral appliance with microneedles is shown in FIGS. 1E, 1F and 1G. This type of drug delivery system not only considers the drug itself but also the tissues the medicament will oppose, the delivery characteristics desired (e.g., slow or fast, etc.) and the length of time it will contact the tissues.

In certain aspects, an oral appliance for delivery of a medicament to an oral cavity is provided, wherein the oral appliance has an exterior and an interior, the interior configured to contour at least a hard tissue, a soft tissue and/or artificial surfaces of the oral cavity, wherein the exterior or interior or both comprise a plurality of microneedles, the microneedles configured for the delivery of the medicament. Useful microneedles for the oral appliances contemplated in this disclosure comprise solid, coated or dissolving microneedles. In various aspects, each microneedle has a base portion, a tip end portion distal to the base portion and a body therebetween, with at least the body holding the medicament. In some embodiments, the body of a microneedle is coated with the medicament, wherein the base portion comprises polyethylene (PE), polypropylene (PP), polytetrafluoroethylene (PTFE), polymethylmethacrylate (PMMA), ethylene vinyl acetate (EVA), polycaprolactone (PCL), polyurethane (PU), polyethylene terephthalate (PET), polyethylene glycol (PEG), polyvinyl alcohol (PVA), poly lactide (PLA), poly lactic-co-glycolic acid (PLGA), and polyglycolide (PGA) or combination thereof.

The body portion of a microneedle can include a viscous material, for example a hydrogel, and the medicament transferred to the skin tissue within the mouth. Other useful viscous materials included in the needle body portion includes at least one selected from a group including carboxymethyl cellulose (CMC), hyaluronic acid, alginic acid, pectin, carrageenan, chondroitin (sulfate), dextran (sulfate), chitosan, polylysine, carboxymethyl chitin, fibrin, agarose, pullulan, polyanhydride, polyorthoester, polyetherester, polyesteramide, poly (butyric acid), poly (valeric acid), polyacrylate, ethylene-vinyl acetate polymer, acrylic substituted cellulose acetate, polyvinyl chloride, polyvinylidene fluoride, poly (vinyl imidazole), chlorosulphonate polyolefins, polyethylene oxide, polyvinylpyrrolidone (PVP), hydroxypropyl methyl cellulose (HPMC), ethylcellulose (EC), hydroxypropyl cellulose (HPC), carboxymethyl cellulose, cyclodextrin, maltose, lactose, trehalose, cellobiose, isomaltose, turanose or lactulose and copolymer of monomers constituting the polymer, and cellulose. In certain embodiments, the microneedles of the oral appliance can be used to deliver a medicament either as part of the needle body portion or as a coating outside the body portion. Medicaments such as lidocaine, mepivacaine, prilocaine, bupivacaine, etidocaine, articaine, procaine, propoxycaine, tetracaine, ropivacaine, butacaine, piperocaine, cocaine, benzocaine, chloroprocaine, proparacaine, and dyclonine can be delivered with the microneedles of the oral appliance.

In various embodiments, the microneedles of the oral appliance are configured to dissolve and comprise (i) a biodegradable polymer resin comprising poly lactic acid (PLA) or poly lactic-co-glycolic acid (PLGA), or (ii) hydroxyethyl starch 7000, hyaluronic acid, polyvinyl alcohol or sucrose. In other embodiments, the microneedles are solid and comprise silicone or ceramic. In many aspects, each microneedle of the oral appliance can have a variable length from about 100 to about 1500 μm.

### Polymeric Matrix Material

The oral appliance contains a polymeric matrix material which can be located at discrete regions or in a channel on the interior surface of the oral appliance. In many embodiments, the polymeric matrix comprises a polymer or a hydrogel. In some embodiments, the polymer includes ethylene glycol dimethacrylate (EGDMA), hydroxypropylcellulose (HPC) or poly(2-hydroxyethyl methacrylate) (pHEMA) or a combination thereof. In some embodiments, the polymer or hydrogel includes from about 80 wt. %, 81%, 82%, 83%, 84%, 85%, 86%, 87%, 88%, 89%, 90%, 91%, 92%, 93%, 94%, 95%, 96%, 97%, 98%, to about 99 wt. % of the polymeric matrix. In other embodiments, the polymer or hydrogel includes from about 80% to about 99% weight/weight (w/w), volume/volume (v/v) or weight/volume (w/v) of the polymeric matrix.

Generally, “hydrogel” refers to a network of polymer chains that are hydrophilic but water insoluble. Hydrogels are sometimes found as colloidal gels in which water is the dispersion medium. Hydrogels are superabsorbent (they can contain over 99% water) natural or synthetic polymers.

In various embodiments, the molecular weight of the hydrogel can be varied as desired. The choice of method to vary molecular weight is typically determined by the composition of the hydrogel (e.g., polymer, versus non-polymer). For example, in various embodiments, when the hydrogel comprises one or more polymers, the degree of polymerization can be controlled by varying the amount of polymer initiators (e.g., benzoyl peroxide), organic solvents or activator (e.g., DMPT), crosslinking agents, polymerization agent, incorporation of chain transfer or chain capping agents and/or reaction time.

In various embodiments, when the hydrogel is polymerized by UV curing, a photoinitiator can be used. Nonlimiting example of commercially available photoinitiators include diethoxyacetophenone (DEAP), dimethoxyphenylacetophenone (Irgacure 651), benzoylcyclohexanol (Irgacure 184), or hydroxydimethylacetophenone (Darocure 1173). In some embodiments, the amount of photoinitiator can vary from about 0.015, 0.020, 0.025, 0.030, 0.035, 0.040, 0.045, 0.050, 0.055, 0.060, 0.065, 0.070, to about 0.075 mL. In other embodiments, the hydrogel can be polymerized by utilizing polymerizing or crosslinking agents, for example, epoxy resins or polyamines or the polymers can be frozen and then thawed to allow polymerization.

Suitable hydrogel polymers may be soluble in an organic solvent. The solubility of a polymer in a solvent varies depending on the crystallinity, hydrophobicity, hydrogen-bonding and molecular weight of the polymer. Lower molecular weight polymers will normally dissolve more readily in an organic solvent than high-molecular weight polymers. A polymeric hydrogel that includes a high molecular weight polymer tends to coagulate or solidify more quickly than a polymeric composition that includes a low-molecular weight polymer. Polymeric hydrogel formulations that include high molecular weight polymers also tend to have a higher solution viscosity than a polymeric hydrogel that includes low-molecular weight polymers. In various embodiments, the molecular weight of the polymer can be a wide range of values. The average molecular weight of the polymer can be from about 1000 to about 10,000,000; or about 1,000 to about 1,000,000; or about 5,000 to about 500,000; or about 10,000 to about 100,000; or about 20,000 to 50,000 g/mol; or about 300 kDa to about 1,000 kDa.

In various embodiments, the hydrogel has an inherent viscosity (abbreviated as “I.V.” and units are in deciliters/gram), which is a measure of the hydrogel's molecular weight and degradation time (e.g., a hydrogel with a high inherent viscosity has a higher molecular weight and may have a longer degradation time).

Typically, when the polymers have similar components but different molecular weights, a hydrogel with a high molecular weight provides a stronger matrix and the matrix takes more time to degrade. In contrast, a hydrogel with a low molecular weight degrades more quickly and provides a softer matrix. In various embodiments, the hydrogel has a molecular weight, as shown by the inherent viscosity, from about 0.10 dL/g to about 1.2 dL/g or from about 0.10 dL/g to about 0.40 dL/g. Other IV ranges include but are not limited to about 0.05 to about 0.15 dL/g, about 0.10 to about 0.20 dL/g, about 0.15 to about 0.25 dL/g, about 0.20 to about 0.30 dL/g, about 0.25 to about 0.35 dL/g, about 0.30 to about 0.35 dL/g, about 0.35 to about 0.45 dL/g, about 0.40 to about 0.45 dL/g, about 0.45 to about 0.55 dL/g, about 0.50 to about 0.70 dL/g, about 0.60 to about 0.80 dL/g, about 0.70 to about 0.90 dL/g, about 0.80 to about 1.00 dL/g, about 0.90 to about 1.10 dL/g, about 1.0 to about 1.2 dL/g, about 1.1 to about 1.3 dL/g, about 1.2 to about 1.4 dL/g, about 1.3 to about 1.5 dL/g, about 1.4 to about 1.6 dL/g, about 1.5 to about 1.7 dL/g, about 1.6 to about 1.8 dL/g, about 1.7 to about 1.9 dL/g, and about 1.8 to about 2.1 dL/g.

In some embodiments, when the polymer materials of the polymeric matrix have different chemistries (e.g., high MW DLG 5050 and low MW DL), the high MW polymer may degrade faster than the low MW polymer. In other aspects, the polymer material of the polymeric matrix has a porosity of from about 60 to about 600 μm.

In various embodiments, the hydrogel can have a viscosity of about 300 to about 5,000 centipoise (cp). In other embodiments, the hydrogel can have a viscosity of from about 5 to about 300 cps, from about 10 cps to about 50 cps, or from about 15 cps to about 75 cps at room temperature. The hydrogel may optionally have a viscosity enhancing agent such as, for example, hydroxypropyl cellulose, hydroxypropyl methylcellulose, hydroxyethyl methylcellulose, carboxymethylcellulose and salts thereof, Carbopol, poly-(hydroxyethylmethacrylate), poly-(methoxyethylmethacrylate), poly(methoxyethoxyethyl methacrylate), polymethylmethacrylate (PMMA), methylmethacrylate (MMA), gelatin, polyvinyl alcohols, propylene glycol, mPEG, PEG 200, PEG 300, PEG 400, PEG 500, PEG 600, PEG 700, PEG 800, PEG 900, PEG 1000, PEG 1450, PEG 3350, PEG 4500, PEG 8000 or combinations thereof. In some aspects, the polymer material (e.g., hydrogel) of the polymeric matrix has a viscosity of from about 50,000 cps to about 1×10⁷ cps at 25° C.

In various embodiments, the hydrogel is made of high molecular weight biocompatible elastomeric polymers of synthetic or natural origin. In other embodiments, the hydrogel material can hold collected biological materials when the hydrogel material is hypo-saturated, saturated, or supersaturated. There are many advantages resulting from using hydrogel as the polymeric matrix for medicament used in the oral appliances described herein. Generally, hydrogel materials provide an effective contact medium for gum compression and for collecting biological materials for diagnosis. The above can hold the sample (e.g., saliva, blood, cells, etc.) when the oral appliance is removed and then the oral appliance can be sent to the lab for testing. Sending out the entire oral appliance to the lab can prevent cross contamination of the patient's hands contaminating the sample collected by the hydrogel. On the other hand, in some embodiments, only the hydrogel polymeric matrix can be removed and then sent out to the lab for testing.

In various embodiments, useful materials for preparing the polymeric matrix for the medicament used in the oral appliances described in this disclosure comprise reactive segmented block copolymers containing hydrophilic domain(s) and showing good surface properties when the block copolymer is covalently bound to substrates containing complimentary functionality. The hydrophilic domain(s) will comprise at least one hydrophilic monomer, such as, HEMA, glyceryl methacrylate, methacrylic acid (“MAA”), acrylic acid (“AA”), methacrylamide, acrylamide, N,N′-dimethylmethacrylamide, or N,N′-dimethylacrylamide; copolymers thereof; hydrophilic prepolymers, such as ethylenically unsaturated poly(alkylene oxide)s, cyclic lactams such as N-vinyl-2-pyrrolidone (“NVP”), or derivatives thereof. Still further examples are the hydrophilic vinyl carbonate or vinyl carbamate monomers. Hydrophilic monomers can be nonionic monomers, such as 2-hydroxyethyl methacrylate (“HEMA”), 2-hydroxyethyl acrylate (“HEA”), 2-(2-ethoxyethoxy)ethyl(methacrylate), glyceryl(meth)acrylate, poly(ethylene glycol(methacrylate), tetrahydrofurfuryl(methacrylate), (methacrylamide), N,N′-dimethylmethacrylamide, N,N′-dimethylacrylamide (“DMA”), N-vinyl-2-pyrrolidone (or other N-vinyl lactams), N-vinyl acetamide, and combinations thereof. Still further examples of hydrophilic monomers are the vinyl carbonate and vinyl carbamate monomers disclosed in U.S. Pat. No. 5,070,215, and the hydrophilic oxazolone monomers disclosed in U.S. Pat. No. 4,910,277. The contents of these patents are incorporated herein by reference. The hydrophilic monomer also can be an anionic monomer, such as 2-methacryloyloxyethylsulfonate salts. Substituted anionic hydrophilic monomers, such as from acrylic and methacrylic acid, can also be utilized wherein the substituted group can be removed by a facile chemical process.

In various embodiments, monomers and/or copolymers useful in preparing the hydrogel polymeric matrix for the oral appliances described in this application include without limitation 2-hydroxyethyl methacrylate (HEMA), ethylene glycol dimethacrylate (EGDMA) and/or poly-2-hydroxyethyl methacrylate (pHEMA) or combinations thereof. Polymers useful in preparing the hydrogel polymeric matrices for the oral appliances described in this application include without limitation poly-2-hydroxyethyl methacrylate (pHEMA), poly(ethylene glycol) diacrylate (PEGDA) or hydroxypropyl cellulose (HPC) or combinations thereof. In some aspects, pHEMA can have various molecular weights, for example, 300,000 Da or 1,000,000 Da. In various aspects, the amount of pHEMA useful for preparing the hydrogels described in this disclosure varies from about 15% to about 50 w/w, v/v or w/v and the amount of EGDMA varies from about 0.001% to about 0.005 w/w, v/v or w/v based on a total weight or a total volume of the hydrogel.

### Medicaments

The oral appliance contains one or more medicaments coated or layered on the polymeric matrix disposed on the interior of the oral appliance at discrete regions or in a continuous channel. The medicament can be disposed on a porous material that can be separated from the oral appliance. Alternatively, the medicament can be disposed on or in the interior of the oral appliance. In some embodiments, the oral appliance has the medicament disposed uniformly throughout the oral appliance.

In various embodiments, some areas of the polymer material of the oral appliance do not contain one or more medicaments, and the polymer material may function to hold or lock a portion of the polymer material in place so that other portions of the polymer material can contact the appropriate target site. Thus, in some embodiments, the polymer material may contain one or more medicaments disposed at discrete regions on the interior surface of the oral appliance. In other embodiments, one or more portions of the oral appliance do not contain any medicament disposed in or on it (e.g., the non-porous regions of the oral appliance). The term “medicament” as used herein is generally meant to refer to any substance that alters the physiology of a patient. The term “medicament” may be used interchangeably herein with the terms “medicine”, “drug” “therapeutic agent”, “therapeutically effective amount”, or “active pharmaceutical ingredient”. It will be understood that a “medicament formulation” may include more than one therapeutic agent, wherein exemplary combinations of therapeutic agents include a combination of two or more medicaments. The medicament can also include cells, where the device (e.g., oral appliance) can be seeded with the cells, for example, gingival cells or gingival tissue, bone cells, cartilage cells or bone tissue so that the device can repair or replace tissue in the treatment area. Other bacterial or other life forms in toto or in part(s) may also be loaded as well as bacterial phages.

The medicament may be in powder, liquid, solid, solution, or suspension (e.g., hydrogel) form and disposed on or impregnated in the oral appliance. In some embodiments, the medicament can be mixed with the polymer from which the oral appliance is made. This may occur during manufacture of the oral appliance or it may occur after the oral appliance is made. For example, on the core polymer material of the oral appliance, the medicament may be layered by solution or suspension layering or powder layering techniques. In solution or suspension layering, the medicament and any inactive ingredients (excipients, binders, etc.) are suspended or dissolved in water or an organic solvent. The resulting liquid is sprayed on the interior surface of the oral appliance to make the polymeric matrix have the desired potency. Solution or suspension layering may be conducted using a wide variety of process techniques, for example, by fluidized bed, Wurster bottom spray techniques, or the like. When the desired potency has been achieved, the polymeric matrix is dried to the desired residual moisture content. Powdered layering involves the application of a dry powder to the oral appliance. The powder may contain the drug, or may include excipients such as a binder, flow aid, inert filler, and the like. In the powder layering technique, a pharmaceutically acceptable liquid, which may be water, organic solvent, with or without a binder and/or excipient, is applied to the polymeric matrix of the oral appliance while applying the dry powder until the desired potency is achieved. When the desired potency has been achieved, the oral appliance may be dried to the desired moisture content.

In various embodiments, the medicament is in liquid form and is capable of diffusing through and within the polymer material. In various embodiments, the liquid medicament may flow or diffuse from one portion of the oral appliance to another portion. In some embodiments, the liquid medicament may not flow or diffuse within the oral appliance. In some embodiments, the liquid medicament is confined within the regions of the oral appliance corresponding to the treatment area. The liquid medicament is not capable of flowing or diffusing into the non-porous regions of the oral appliance. In some embodiments, the liquid medicament may flow or diffuse into the non-porous regions; however, the medicament cannot easily flow or diffuse out of the non-porous regions. In various aspects, the medicament located at discrete regions in the interior surface of the oral appliance is free of hydrogen peroxide.

Examples of medicaments include, but are not limited to, anti-inflammatory agents, antimicrobial agents (e.g., antiviral, antibacterial, antibiotic, antifungal agents, antiseptic agents, etc.), tissue and bone growth factors, pain management medication (e.g., analgesics, anesthetics, cannabidiol (CBD) etc.), tooth whitening agents, breath fresheners, anticalculus agents, anticeptics, anticaries agents, nutrients, vitamins, minerals, herbal products, probiotics, immunologic agents, astringents, an ointment or liniment or mixtures thereof.

Suitable anti-inflammatory agents to treat and/or reduce inflammation include steroidal and/or non-steroidal anti-inflammatories. Exemplary anti-inflammatory agents include by way of example and not limitation, alclofenac; alclometasone dipropionate; algestone acetonide; alendronate sodium; alpha amylase; amcinafal; amcinafide; amcinonide; amfenac sodium; amiprilose hydrochloride; anakinra; anirolac; anitrazafen; apazone; balsalazide disodium; beclomethasone diproprionate; bendazac; benoxaprofen; benzydamine hydrochloride; betamethasone; bromelains; broperamole; budesonide; carprofen; cicloprofen; cintazone; cliprofen; clobetasol propionate; clobetasone butyrate; clopirac; cloticasone propionate; cormethasone acetate; cortisone acetate; cortodoxone; deflazacort; desonide; desoximetasone; dexamethasone dipropionate; diclofenac potassium; diclofenac sodium; diflorasone diacetate; diflumidone sodium; diflunisal; difluprednate; diftalone; dimethyl sulfoxide; drocinonide; endrysone; enlimomab; enolicam sodium; epirizole; etodolac; etofenamate; felbinac; fenamole; fenbufen; fenclofenac; fenclorac; fendosal; fenpipalone; fentiazac; flazalone; fluazacort; fludrocortisone; flufenamic acid; flumizole; flunisolide acetate; flunixin; flunixin meglumine; fluocinonide; fluocinolone acetonide; fluocortin butyl; fluorometholone acetate; fluquazone; flurandrenolide; flurbiprofen; fluretofen; fluticasone propionate; furaprofen; furobufen; halcinonide; halobetasol propionate; halopredone acetate; hydrocortisone; ibufenac; ibuprofen; ibuprofen aluminum; ibuprofen piconol; ilonidap; indomethacin; indomethacin sodium; indoprofen; indoxole; intrazole; isoflupredone acetate; isoxepac; isoxicam; ketoprofen; lofemizole hydrochloride; lomoxicam; loteprednol etabonate; meclofenamate sodium; meclofenamic acid; meclorisone dibutyrate; medrysone; mefenamic acid; mesalamine; meseclazone; methylprednisolone suleptanate; momiflumate; nabumetone; naproxen; naproxen sodium; naproxol; nimazone; nilutamide; olsalazine sodium; orgotein; orpanoxin; oxaprozin; oxyphenbutazone; pamidronate disodium; paramethasone; paranyline hydrochloride; pentosan polysulfate sodium; phenbutazone sodium glycerate; pirfenidone; piroxicam; piroxicam cinnamate; piroxicam olamine; pirprofen; prednazate; prednisolone; prifelone; prodolic acid; proquazone; proxazole; proxazole citrate; rimexolone; romazarit; salcolex; salnacedin; salsalate; sanguinarium chloride; seclazone; sermetacin; sudoxicam; sulindac; suprofen; talmetacin; talniflumate; talosalate; tebufelone; tenidap; tenidap sodium; tenoxicam; tesicam; tesimide; tetrydamine; tiopinac; tixocortol pivalate; tolmetin; tolmetin sodium; triamcinelone; triclonide; triflumidate; zidometacin; zomepirac sodium or combinations thereof.

Anti-inflammatory agents include steroidal agents or glucocorticosteroids. Phospholipase A2 (“PLA2”) is a lipolytic enzyme that has been implicated as a possible mediator of inflammation. Specifically, PLA2 hydrolyses the 2-acyl position of glycerophospholipids, liberating free-fatty acids, mainly arachidonic acid. Subsequently, it is believed that arachidonic acid is converted into a variety of proinflammatory cicosanoids. Glucocorticosteroids are known to stop or reduce the suggested mechanisms of inflammation that involves the activation of the arachidonic acid cascade, which results in the liberation of a variety of proinflammatory eicosanoids by inducing lipocortin that inhibits PLA2. This provides a significant advantage over non-steroidal anti-inflammatory agents that enter the cascade much later.

Suitable glucocorticosteroids include, but are not limited to, alclometasone diproprionate, alendronate sodium, amcinonide, beclomethasone diproprionate, betamethasone, budesonide, clobetasol propionate, cortisone, dexamethasone, diflorasone diacetate, hydrocortisone, fludrocortisone; flunisolide acetate, fluocinolone acetonide, fluocinonide, fluorometholone acetate, flurandrenolide, halcinonide, medrysone; methylprednisone suleptanate, pamidronate, paramethasone, prednisolone, nilutamide, triamcinelone, or combinations thereof.

Dexamethasone is of particular interest for use as an anti-inflammatory to treat orofacial diseases. Besides its anti-inflammatory property, dexamethasone can be delivered to up-regulate certain enzyme activities. Specifically, dexamethasone can be used to increase or up-regulate alkaline phosphotase activity in regenerating human periodontal cells.

Exemplary anti-infective agents to treat infection include by way of example and not limitation, antimicrobial agents; quinolones and in particular fluoroquinolones (e.g., norfloxacin, ciprofloxacin, lomefloxacin, ofloxacin, levofloxacin, delafloxacin, etc.), aminoglycosides (e.g., gentamicin, tobramycin, etc.), glycopeptides (e.g., vancomycin, etc.), lincosamides (e.g., clindamycin), cephalosporins (e.g., first, second, third generation) and related beta-lactams, macrolides (e.g., azithromycin, erythromycin, clarithromycin, etc.), nitroimidazoles (e.g., metronidazole), penicillins, polymyxins, tetracyclines, triamcinolone, triclosan or combinations thereof.

Other exemplary antimicrobial agents include, by way of illustration and not limitation, acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbapenem, carbenicillin disodium; carbenicillin indanyl sodium; carbenicillin phenyl sodium; carbenicillin potassium; carumonam sodium; cefaclor, cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; monobactams; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; zorbamycin; or combinations thereof.

Exemplary analgesics include, but are not limited to, acetaminophen; alfentanil hydrochloride; aminobenzoate potassium; aminobenzoate sodium; anidoxime; anileridine; anileridine hydrochloride; anilopam hydrochloride; anirolac; antipyrine; aspirin; benoxaprofen; benzydamine hydrochloride; bicifadine hydrochloride; brifentanil hydrochloride; bromadoline maleate; bromfenac sodium; buprenorphine hydrochloride; butacetin; butixirate; butorphanol; butorphanol tartrate; carbamazepine; carbaspirin calcium; carbiphene hydrochloride; carfentanil citrate; ciprefadol succinate; ciramadol; ciramadol hydrochloride; clonixeril; clonixin; codeine; codeine phosphate; codeine sulfate; conorphone hydrochloride; cyclazocine; dexoxadrol hydrochloride; dexpemedolac; dezocine; diflunisal; dihydrocodeine bitartrate; dimefadane; dipyrone; doxpicomine hydrochloride; drinidene; enadoline hydrochloride; epirizole; ergotamine tartrate; ethoxazene hydrochloride; etofenamate; eugenol; fenoprofen; fenoprofen calcium; fentanyl citrate; floctafenine; flufenisal; flunixin; flunixin meglumine; flupirtine maleate; fluproquazone; fluradoline hydrochloride; flurbiprofen; hydromorphone hydrochloride; ibufenac; ibuprofen; indoprofen; ketazocine; ketorfanol; ketorolac and ketorolac tromethamine; letimide hydrochloride; levomethadyl acetate; levomethadyl acetate hydrochloride; levonantradol hydrochloride; levorphanol tartrate; lidocaine, bupivacaine, lofemizole hydrochloride; lofentanil oxalate; lorcinadol; lomoxicam; magnesium salicylate; mefenamic acid; menabitan hydrochloride; meperidine hydrochloride; meptazinol hydrochloride; methadone hydrochloride; methadyl acetate; methopholine; methotrimeprazine; metkephamid acetate; mimbane hydrochloride; mirfentanil hydrochloride; molinazone; motrin; morphine sulfate; moxazocine; nabitan hydrochloride; nalbuphine hydrochloride; nalmexone hydrochloride; namoxyrate; nantradol hydrochloride; naproxen; naproxen sodium; naproxol; nefopam hydrochloride; nexeridine hydrochloride; noracymethadol hydrochloride; ocfentanil hydrochloride; octazamide; olvanil; oxetorone fumarate; oxycodone; oxycodone hydrochloride; oxycodone terephthalate; oxymorphone hydrochloride; pemedolac; pentamorphone; pentazocine; pentazocine hydrochloride; pentazocine lactate; phenazopyridine hydrochloride; phenyramidol hydrochloride; picenadol hydrochloride; pinadoline; pirfenidone; piroxicam olamine; pravadoline maleate; prodilidine hydrochloride; profadol hydrochloride; propiram fumarate; propoxyphene hydrochloride; propoxyphene napsylate; proxazole; proxazole citrate; proxorphan tartrate; pyrroliphene hydrochloride; remifentanil hydrochloride; salcolex; salethamide maleate; salicylamide; salicylate meglumine; salsalate; sodium salicylate; spiradoline mesylate; sufentanil; sufentanil citrate; talmetacin; talniflumate; talosalate; tazadolene succinate; tebufelone; tetrydamine; tifurac sodium; tilidine hydrochloride; tiopinac; tonazocine mesylate; tramadol hydrochloride; trefentanil hydrochloride; trolamine; veradoline hydrochloride; verilopam hydrochloride; volazocine; xorphanol mesylate; xylazine hydrochloride; zenazocine mesylate; zomepirac sodium; zucapsaicin or combinations thereof.

Antifungal agents that can be used in the oral appliance include, but are not limited to, nystatin, clotrimazole, griseofulvin, ketoconazole, itraconazole, fluconazole, terbinafine, or a combination thereof.

Exemplary antiseptics include chlorhexidine, chlorhexidine gluconate, hexetidine, hydrogen peroxide, sodium hypochlorite, cetylpyridinium chloride, triclosan, methyl salicylate, povidone-iodine and the like. Astringents, for example, zinc chloride are also useful medicaments for the oral appliances described in this disclosure.

Oral appliances are provided that can deliver medicaments and/or tissues to at least a portion of the teeth and/or soft tissues inside the oral cavity in a three-dimensional way. One advantage of the oral appliance is that it is custom made to fit only one patient. As used herein a “custom fit” oral appliance refers to an oral appliance prepared to correspond to at least a portion of the teeth or all of the teeth and soft tissues of a specific patient. Typically, the custom fit appliance is prepared by a dental care professional (e.g., dentist, oral surgeon, medical doctor, other health care professional, manufacturer, etc.). The custom fit oral appliance can be made from an impression mold or using an analog or digital image capturing device. The oral appliance provided by this disclosure is not a boil-and-bite prefabricated device or a stock oral appliance, which can be manipulated by the consumer himself/herself with fingers to shape it against the teeth and gums, but which cannot possibly be shaped to properly align the medication with the proper geographic anatomy.

In some embodiments, the oral appliance can contain medicament separately in a cargo area or sponge or placed as a liquid in the oral appliance. The oral appliances disclosed herein are custom fit, disposable, and manufactured in one continuous step, pre-loaded with medicament in or on at least a portion of the interior and/or exterior surfaces of the appliance and can deliver medicaments or graft tissues three dimensionally. In some embodiments, the oral appliance can be transparent. In other embodiments, the oral appliance can be translucent of opaque. Still another advantage of the oral appliance is that, in various embodiments, it can be easily manufactured and is comfortable for the patient to use. Other advantages of the oral appliances provided by this disclosure include greater efficacy over conventional oral therapies based on two dimensional systems, user convenience, enhanced patient compliance, lower dosage requirements, less dilution of medicament and enhanced applied pressure to gums.

In one embodiment, there is an oral appliance for delivering a medicament to at least a portion of teeth and/or soft tissue areas inside an oral cavity, the oral appliance comprising an interior surface having a medicament disposed in or on at least a portion of and/or all of the interior surface of the oral appliance, the interior surface being formed to fit contours of at least the portion of the teeth and/or soft tissue, including inflamed soft tissue areas inside the oral cavity and being configured for holding the medicament in contact with at least the portion of the teeth and/or soft tissue areas inside the oral cavity to deliver the medicament thereto.

The soft tissue of the inside of the mouth includes, but is not limited to, any soft tissue adjacent or between the teeth including, but not limited to, the papilla, tissue of the upper and lower dental arches, marginal gingiva, gingival sulcus, inter-dental gingiva, gingival gum structure on lingual and buccal surfaces up to and including the muco-gingival junction and/or the upper palate and/or the floor of the mouth.

In various embodiments, the soft tissue area inside the oral cavity includes the muco-buccal folds, hard and soft palates, lining mucosa, the tongue (one or all surfaces of the tongue) and/or attached gingival tissue, all of which may occasionally become inflamed as caused by any number of conditions. In various embodiments, the oral appliance receives one or more teeth including one or more molars, premolars, incisors, cuspids, tooth implant, or combination or portions thereof. In other embodiments, the medicament contained in the oral appliance can be disposed anywhere in or on the interior or exterior surface of the oral appliance adjacent to the gum and/or other soft tissue areas of the oral cavity including the front, back, occlusal surfaces of one or more teeth.

In various embodiments, the oral appliance may contain more than one medicament. However, in another embodiment, combination therapy will involve use of a single, safe and effective amount of the medicament. For example, the method may further comprise subsequently administering one or more additional oral appliances, each containing a medicament that is different from the medicament contained in the earlier oral appliance. In this way, a series of customized treatment regimens can be provided to the patient. This provides for a “mix and match” medicament regimen with dose adjustment capability and provides the added advantage of allowing the health professional complete control to administer only those medicaments at the desired strength believed to be appropriate for the disease or condition being treated to a particular individual.

The amount of medicament contained within the oral appliance, will vary widely depending on the effective dosage required and rate of release from the polymer material and the length of the desired delivery interval. The dosage administered to the patient can be single or multiple doses and will vary depending upon a variety of factors, including the agent's pharmacokinetic properties, patient conditions and characteristics (e.g., sex, age, body weight, health, size, etc.), extent of symptoms, concurrent treatments, frequency of treatment and the effect desired. These factors can readily be determined by those of ordinary skill in the art.

In various embodiments, the polymer material of the oral appliance is designed to release the medicament as a bolus dose of the medicament, a single dose of the medicament, or multiple doses of the medicament all preloaded with a specific dosage at the manufacturing facility.

In some embodiments, the medicament described herein is in the oral appliance in an amount of from about 0.01, 0.02, 0.03, 0.04, 0.05, 0.06, 0.07, 0.08, 0.09, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, to about 50% by weight of the oral appliance.

In some embodiments, the medicament can be disposed anywhere in or on the interior or exterior surface of the oral appliance adjacent to the gum and/or other soft tissue areas of the oral cavity including the buccal, lingual, palatal, mesial, distal, occlusal surfaces of one or more teeth. Some portions of the oral cavity, for example artificial surfaces of dental crowns or bridges that do not require the medicament are sealed with the non-porous material which can be a coating, cross-linked with porosity reducing agent or comprise non-porous material such that the medicament cannot penetrate said portions. In some embodiments, the medicament may be disposed in or may enter the non-porous region. However, the medicament disposed in the non-porous region will not release the medicament or will release the medicament at a reduced rate.

### Methods of Making the Oral Appliance

In various embodiments, a method of making an oral appliance for delivering a medicament to an oral cavity is provided. The method includes providing an oral appliance having an interior surface and/or exterior surface, the interior surface and/or exterior surface configured to contour a treatment area and deliver medicament thereto, the treatment area comprising soft tissue, hard tissue or a combination of soft tissue and hard tissue, at least a portion of the interior surface and/or exterior surface of the oral appliance being about as soft or as hard to match the softness or hardness of the treatment area; and dispensing the medicament in the interior surface of the oral appliance for delivery to the oral cavity. In some embodiments, the oral appliance comprises an exterior surface and all or a portion of the interior surface and/or exterior surface contains a medicament, the interior surface and/or exterior surface having a softness to match the treatment surface it contours. In other embodiments, the oral appliance comprises an exterior surface and all or a portion of the interior surface contains a medicament, the interior surface and/or exterior surface having a hardness to match the treatment surface it contours.

The oral appliance is custom made to fit a specific patient. The custom-made oral appliance may be prepared by a health care professional including, but not limited to a dentist, oral surgeon, medical doctor, technician or manufacturer. The oral appliance can be made from an impression mold, or by using an analog or digital image capturing device. The oral appliance disclosed herein is not a boil and bite prefabricated device or a stock oral appliance which can be manipulated by the consumer himself/herself with fingers to shape it against the teeth and gums. The oral appliance disclosed herein is custom fit, disposable, and monolithic and is pre-loaded with medicament into or onto, in some aspects, at least a portion of the interior and/or exterior surfaces of the appliance and can deliver medicament as more particularly described in U.S. patent application Ser. No. 15/895,554 to Peter J. Zegarelli, filed on Feb. 13, 2018. The entire disclosure of this application is incorporated herein by reference into the present application.

In some embodiments, the dental practitioner identifies the region that requires the medicament and the region that does not require the medicament, for example an artificial surface, such as a dental crown or bridge or an implant. The region that requires the medicament is made with porous material infused with medicament and the region that does not require the medicament is made with non-porous materials.

In some embodiments, the entire oral appliance, the porous material, or discrete regions of the oral appliance includes or is manufactured to include carbon foam, polymer(s), or a combination thereof. The foam can be a carbon foam lattice, such as carbon resin DPR 10 (Carbon 3D, Inc. C.A.). These polymer materials can be printed, for example, by Carbon 3D printers.

The carbon foam, and/or polymer(s), can affect the application and/or release of the medicament. In some embodiments, the entire oral appliance is made from carbon foam or polymer(s), and the density of the carbon foam or polymer(s) vary from very dense regions which create the non-porous regions of the oral appliance, to less dense regions which create the porous regions of the oral appliance. The very dense, non-porous regions prevent the release of the medicament.

The carbon foam or polymer(s) allow the porosity of the oral appliance to be controlled. For example, the oral appliance can be 3D printed with carbon foam or polymer(s) and the areas not including the gum line can be printed densely where there are little to no open cell, lattice or honeycomb configurations present. However, the gum line area will be printed with the carbon foam or polymer(s) in a less dense manner where open cell, lattice or honeycomb configurations are present to allow influx, or allow release of antimicrobial or other substances to the gum line area. In some embodiments, some areas of the oral appliance can be printed virtually as a solid, and other areas of the oral appliance can be printed as a semi-solid.

In some embodiments, when the oral appliance is made by 3D printing and different polymers having a different density are used, the printer can be instructed to print the low-density polymer at discrete regions of the oral appliance, which will be the porous region of the oral appliance. The printer can be instructed to print a higher density of polymer on the oral appliance to make discrete non-porous regions of the oral appliance.

It is to be understood that the polymerizable liquid is reactive to irradiation such as light (e.g., ultraviolet (UV) light) and the polymerizable liquid can contain photoreactive or photocurable groups for such reactivity to take place. The UV light can be controlled by a computer and the light will irradiate the polymerizable liquid for polymerization.

A method of treating a diseased tissue of an oral cavity is also provided. The method of treatment comprises providing an oral appliance for delivering a medicament to an oral cavity, the oral appliance having an interior surface and exterior surface, the interior surface and/or exterior surface configured to contour a treatment area and deliver medicament thereto, the treatment area comprising soft tissue, hard tissue or a combination of soft tissue and hard tissue, at least a portion of the interior surface and/or exterior surface of the oral appliance being about as soft or as hard to match the softness or hardness of the treatment area, the interior surface and/or exterior surface having a medicament disposed at discrete regions of the interior surface and/or exterior surface for delivering the medicament to the diseased tissue. In some embodiments, the oral appliance comprises an exterior surface and all or a portion of the interior surface contains a medicament, the interior surface and/or exterior surface having a softness to match the treatment surface it contours. In other embodiments, the oral appliance comprises an exterior surface and all or a portion of the interior surface contains a medicament, the interior surface having a hardness to match the treatment surface it contours.

In some embodiments, the porous material can be a hydrogel that can have optimal adhesion properties to the underlying tray/shell/scaffold. In some embodiments, the hydrogel can adhere and bind to the underlying tray/shell/scaffold and have complementary material and structural properties to enhance drug delivery. For example, the hydrogel, when cured, should not delaminate when subjected to the compressive, stretching, and shearing forces of the mouth. Further, when cured or otherwise bound together, the adhesion of the tray/shell/scaffold and the hydrogel should not affect the other properties of the tray/shell/scaffold, which have been formulated to enhance delivery of the medicament.

Having now generally described the invention, the same may be more readily understood through the following reference to the following examples, which are provided by way of illustration and are not intended to limit the present invention unless specified.

### EXAMPLES

The examples below are contemplated and an oral appliance is designed using various parameters of material sciences to enhance the delivery of medicaments to the oral cavity by manipulating the physical and chemical properties of the shell or scaffold of the oral appliance and the hydrogel such that they complement each other in the delivery of the medicaments, considering, among other things, the medicaments properties, the characteristics of the disease being treated or prevented, the desired method of delivery, the comfort of the patient, the time of dosing desired, the diffusion rate, the sustained application of medicaments, the targeted tissue to be administered to and the characteristics of those tissues. Therefore, one can change the material and structural properties of the oral appliance in coordination to: the target disease, target tissue, whether mucosal or transmucosal, medicament, and release type (e g, immediate release, delayed release, etc.).

The medicament used can be based on the Biopharmaceutics Classification System (BCS), which is a system to differentiate the medicaments on the basis of their solubility and permeability with a focus on the medicament solubility and the target oral tissue permeability. The hydrogel can be designed according to these parameters.

| Class | Features | Medicament Examples |
|---|---|---|
| I | High permeability | paracetamol, metoprolol, |
| | High solubility | theophylline |
| II | High permeability | atovaquone, carbamazepine |
| | Low solubility | danazol, glibenclamide, |
| | | griseofulvin, ketoconazole, |
| | | troglitazone |
| III | Low permeability | acyclovir, atenolol |
| | High solubility | cimetidine, ranitidine |
| IV | Low permeability | chlorothiazide, furosemide |
| | Low solubility | |

### Example 1

Target disease: Periodontal disease
Target tissue: Gingiva (gumline)

### Mucosal.

Medicament: chlorhexidine (antiseptic or antimicrobial).
Release type: Relatively slow (10-30 minutes).

Chlorhexidine is soluble and delivery can be slow mucosal delivery. The hydrogel is at the gumline of the oral appliance. The hydrogel is specified to have a certain viscosity, mechanical properties (e.g., elasticity, strength), medicament release profile, etc. The oral appliance is designed mostly around comfort to increase patient compliance. So, it has an elasticity and softness. In summary, we are changing the material properties of the oral appliance to enhance drug delivery given the parameters at hand.

### Example 2

Target disease: Oral mucositis.
Target tissue: Cheeks, tongue, lips.

### Mucosal.

### Medicament: Analgesic.

Release type: Slow (hours).

The oral appliance is designed considering that the solubility of the medicament in water affects the type of hydrogel used. Since it is a mucosal medicament delivery system, there is less concern about getting the analgesic medicament through the tissue into the systemic circulation (more of a safety concern than efficacy), as the design will be on local delivery in the oral cavity. The hydrogel will also be customized for the slow release type (smaller pores/higher cross-linking density, different viscosity, or formulation, etc.). The oral appliance will have hydrogel on the buccal/labial side as well as lingual/palatal side to get the medicament to the target tissue. Since we are treating oral mucositis, the oral appliance can be soft so as not to irritate the tissue, and elastic so that it retains its shape. The oral appliance will be designed around providing comfort to the patient.

### Example 3

For xerostomia, the same structure as Example 2 can be followed. Slow-release a lubricant/moisturizer to the oral mucosa, the oral appliance's material properties to change based on the disease and the progress of treatment.

While particular embodiments of the present disclosure have been shown and described, it will be appreciated by those skilled in the art that, based upon the teachings herein, changes and modifications may be made without departing from this disclosure and its broader aspects and, therefore, the appended claims are to encompass within their scope all such changes and modifications as are within the true spirit and scope of this disclosure. The true spirit and scope is considered to encompass devices and processes, unless specifically limited to distinguish from known subject matter, which provide equivalent functions as required for interaction with other elements of the claims and the scope is not considered limited to devices and functions currently in existence where future developments may supplant usage of currently available devices and processes yet provide the functioning required for interaction with other claim elements.

1. An oral appliance for delivering a medicament to an oral cavity, the oral appliance having an interior surface and an exterior surface, the interior surface and/or exterior surface configured to contour a treatment area and deliver medicament thereto, the treatment area comprising soft tissue, hard tissue or a combination of soft tissue and hard tissue, at least a portion of the interior surface and/or exterior surface of the oral appliance being about as soft or as hard to match the softness or hardness of the treatment area.

2. The oral appliance of claim 1, wherein all or a portion of the interior surface contains a medicament, the interior surface having a softness to match the treatment surface it contours.

3. The oral appliance of claim 1, wherein all or a portion of the exterior surface contains a medicament, the exterior surface having a hardness to match the treatment surface it contours.

4. The oral appliance of claim 1, wherein the hard tissue of the oral cavity comprises at least a tooth and/or other bony structure of the oral cavity.

5. The oral appliance of claim 4, wherein the oral appliance has a modulus of elasticity of from about 1330 to about 1650 MPa to match the hardness of the hard tissue of the oral cavity.

6. The oral appliance of claim 1, wherein the soft tissue of the oral cavity comprises muco-buccal folds, soft palate, lining mucosa, buccal and labial mucosa, attached and unattached gingival tissue, tongue and floor of mouth tissue.

7. The oral appliance of claim 6, wherein the oral appliance has a modulus of elasticity of from about 2.4 to about 19.8 MPa to match the softness of the soft tissue of the oral cavity.

8. The oral appliance of claim 1, wherein the oral appliance has a modulus of elasticity from about 0.01 to about 19 GPa to match the softness of the soft tissue of the oral cavity.

9. The oral appliance of claim 2, wherein the interior surface comprises a polymeric matrix disposed at discrete regions inside the interior surface.

10. The oral appliance of claim 9, wherein the polymeric matrix comprises a hydrogel having a modulus of elasticity from about 8 to about 60 kPa.

11. The oral appliance of claim 10, wherein the hydrogel is a soft hydrogel and has a modulus of elasticity from about 8 kPa.

12. The oral appliance of claim 10, wherein the hydrogel is a hard hydrogel having a modulus of elasticity (i) from about 10 kPa to about 50 kPa; or (ii) from about 50 kPa to about 60 kPa.

13. The oral appliance of claim 11, wherein the medicament comprises an anticariogenic agent, an antimicrobial or a combination thereof.

14. (canceled)

15. The oral appliance of claim 1, wherein the medicament is configured to contact at least a portion of an inflamed tissue of the oral cavity including at least a portion of a subgingival space when the oral appliance is worn to cause delivery of the medicament to at least the portion of the subgingival space of the inflamed tissue.

16. The oral appliance of claim 10, wherein the hydrogel seals an entrance of a gingival crevice or periodontal pocket of a portion of an inflamed tissue of the oral cavity as a portion of the hydrogel exudes from the oral appliance and contacts the entrance of the gingival crevice or periodontal pocket to reduce or prevent contamination of the gingival crevice or periodontal pocket by oral fluids.

17.-22. (canceled)

23. The oral appliance of claim 9, wherein the polymeric matrix has a viscosity of from about 50,000 cps to about 1×10⁷ cps at 25° C.

24. The oral appliance of claim 9, wherein the polymeric matrix has a porosity of from about 60 to about 600 μm.

25.-28. (canceled)

29. An oral appliance for delivering a medicament to an oral cavity, the oral appliance having an exterior and an interior, the interior configured to contour at least a hard tissue, a soft tissue and/or artificial surfaces of the oral cavity, wherein the exterior or interior or both the exterior and interior comprise a plurality of microneedles configured for delivering the medicament.

30. The oral appliance of claim 29, wherein the microneedles comprise solid, coated or dissolving microneedles.

31.-34. (canceled)

35. The oral appliance of claim 29, wherein each microneedle has a length from about 100 to about 1500 μm.

36.-77. (canceled)
